(19) United States
(12) Patent Application Publication
(10) Pub. No.: US 2017/0023446 A1
(43) Pub. Date: Jan. 26, 2017

(54) METHOD AND APPARATUS FOR ACQUIRING BLOOD FOR TESTING

(71) Applicant: Neoteryx, LLC., Torrance, CA (US)

(72) Inventors: Gijsbertus G. Rietveld, Torrance, CA (US); Stuart Kushon, Torrance, CA (US); Monika M. Kansal, Torrance, CA (US)

(21) Appl. No.: 15/048,859

(22) Filed: Feb. 19, 2016

Related U.S. Application Data
(60) Provisional application No. 62/118,982, filed on Feb. 20, 2015.
(60) Provisional application No. 62/143,696, filed on Apr. 6, 2015.

(51) Int. Cl. G01N 1/10; G01N 33/49; A61B 5/15

(52) U.S. Cl. CPC G01N 1/10; A61B 5/150358; A61B 5/150755; G01N 33/49; G01N 2001/1056

(57) ABSTRACT

A blood sampling device is provided having holder with a manipulating end and an absorbent probe on the opposing end. The probe is made of pyrolyzed porous carbon sized to directly absorb a predetermined volume of liquid, preferably biological fluid, in a predetermined amount of time. Shapes for absorbent probes of differing materials are provided.

### CROSS-REFERENCE TO RELATED APPLICATIONS

The application claims the benefit under 35 U.S.C. §119(e) to Provisional Patent Application No. 62/118,982 filed Feb. 20, 2015, and Provisional Patent Application No. 62/143,696 filed Apr. 6, 2015, the entire contents of which are incorporated herein by reference.

### BACKGROUND OF THE INVENTION

This application relates to a method apparatus for sampling blood for use in testing for either research or for diagnostic use.

Multiple blood samples are used for clinical trials for pharmacokinetic analyses. These samples are often collected by sampling whole blood freezing and then processing the frozen blood later. Frozen blood requires a 200-250 ul sample of blood to be taken. This sample size limits the number of time-points which can be taken from a single animal due to the limited blood volume of small animals such as rats. Furthermore small volumes of blood samples are desired when dealing with critically ill patients. Moreover, there are high costs involved with the freezing transportation and processing of whole blood.

Blood samples are also collected using a bloodspot technique which requires smaller sample volumes, typically 45-60 ul for humans and 15 ul for rats, although evolving analytical techniques are using samples using 10-15 ul of human blood and smaller. Referring to FIG. 1, samples are taken from the subject usually by ‘finger pricking’ the individual and then sampling the evolved blood using a glass capillary 5. Once a desired quantity of blood is taken (45-60 ul) then the blood from the capillary 5 is carefully transferred to a ‘blood spot card’ 7 such as Whatman's FDA Eulte, using 15 ul aliquots spots across four spots. Care must be taken not to contaminate the card and not to touch the card with the capillary except for the pre-designated portions where the sample is to be deposited. After blood is taken and spotted, a known concentration of an internal standard is sprayed onto the spotted card and then accurately punching disks (2-6 mm diameter) out of the blood spot or multiple blood spots. Once sampling is complete, the cards 7 are dried in air before transferring or mailing to labs for processing. Because the blood is dried, not only do some enzymatic processes cease preventing further breakdown before testing or during storage, but dried blood is not considered hazardous and no special precautions need be taken in handling or shipping. Once at the analysis site, circular discs containing the dried blood are punched out of the card and the internal standard and drugs (and/or metabolite) are extracted from the disks into a supernatant which is then analyzed usually by liquid chromatography mass spectrometry.

When a card is used for direct sample collection from a wound (e.g. a neonatal heel prick or a finger prick) there is risk for collection of too much blood on the card which will lead to an overlapping of samples from the spots. Additionally, if blood flow is insufficient a non-homogenous sample can be collected (multiple small spots instead of a single large spot). This will lead to difficulty in obtaining a sub-punch from the card that is representative of the entire spot. Additionally, various chemical treatments of card materials can lead to separation of the PCV and serum during the drying process leading to non-homogenous sampling.

There are drawbacks, however, to the downstream processing of blood spots. One is in the area of sample quantitation. It is difficult to sample precise volumes using traditional glass capillaries, particularly directly from an animal or patient blood bolus. Air bubbles in capillaries can result in different capillary volumes being deposited on the cards, leading to different volumes when the card is punched. While use of micropipettes (15 ul sample) can successfully create accurate spot volumes in carefully controlled settings, in practice these have proven to be unreliable.

Another drawback with the punching technique is that it relies on a constant sample viscosity in the expectation that the sample will spread uniformly on the sample card. A constant viscosity results in blood spot diameters remaining constant when equal volume samples are administered to the cards. Unfortunately, viscosity varies significantly because of differing hematocrit (Ht or HCT) or packed cell volume (PCV) levels in the blood. Samples with high hematocrit levels form smaller diameter spots on the bloodspot papers, leading to different concentrations of blood within the fixed diameter of the spots sampled. PCV levels are believed to show around a 45% variance in spot diameters. As internal standards are sprayed onto the spotted blood this could result in a 45% error in quantitation. A further problem is that the blood is placed in marked areas on the cards, but often the person sampling the blood misses the mark and blood goes outside the marked area, making it difficult to accurately locate the circular punch over the blood spot. Even if the blood spot is centered in the card, the person punching the card may not center the punch, resulting in variable sample size. Further, the punching often shears the card and that often shakes dried blood loose, and if the punch cuts across a portion of the blood spot that also causes dried blood to be ejected into the air or work area.

Moreover, the blood spots are placed on rectangular cards which are difficult to manipulate by automated equipment, thus requiring extensive, expensive and time consuming manual handling and processing. Automated handling equipment can be acquired for the specially shaped cards, but it is custom made, expensive, and of limited application.

Absorbent tips have been developed by Porex Corporation to acquire fluids as described in U.S. Pat. Nos. 8,852,122 and 8,920,339 and in patent application Ser. No. 13/668,062 filed Nov. 2, 2012. Holders for those absorbent tips have also been developed as described in those patents and patent applications. But a need for improved, absorbent tips of alternative materials remains unfilled, in particular as the material from the Porex patents is formed by sintering which can affect the shape and functioning of the formed tip.

There is thus a need for an improved method and apparatus for use in blood sampling that reduces or eliminates one or more of the above errors and difficulties.

### SUMMARY

A device is provided that is suitable as a quantitative sampling tool for biological liquids, preferably blood, and is preferably made of a pyrolyzed carbon material advantageously involving a chemical reaction forming carbon-to-carbon bonds and creating an open-cell material. As used herein, liquids and fluids will be used interchangeably but both terms refer to liquids, not gases. The resulting porous carbon micro particles may be formed in a variety of shapes and strengths suitable to a variety of uses. A variety of high-carbon content precursors are believed suitable to form the porous carbon micro particles, including cellulose, sugars, polymers, hydrocarbons, amylose, amylopectin, etc. Carbonization temperature from about 250° C. to about 1500° C. are believed suitable in an environment designed to deplete non-carbon constituents, which may include oxygen or which may be inert (e.g., nitrogen) if the precursor material has a sufficiently high carbon content and sufficiently low impurity content, but with the final pyrolysis forming the porous carbon product occurring without oxygen. A low rate of temperature increase is believed to result in larger retention of the original structure and shape but with lower strength, while a high rate of temperature increase is believed to cause structural collapse and consolidation as the carbon-carbon bonds chemically form and non-carbon materials are burned off in the pyrolytic reaction. The rate of temperature increase or temperature ramp is believed to affect porosity, with lower ramp rates generally increasing porosity but decreasing strength while higher ramp rates generally decrease porosity but increase strength. The pore size of the material is preferably about 10-40 microns so that capillary action draws the fluid into the material and retains it. But the pore size could vary depending on the viscosity of the fluid being absorbed by the material.

The pyrolyzed porous carbon material is advantageously formed in elongated preliminary strips and then cut into a final shape or further formed into a final size or shape suitable for use or for a particular holder. The pyrolyzed porous carbon material may be formed around a handling stem for ease of handling and use. Alternatively, the precursor material may be pyrolyzed in molds having cavities shaped to form the desired shape for the particular use of the material, including shapes formed around a handling stem. Because of the potentially high temperatures the molds may require a metal support with a high-temperature resistant liner made of graphite, ceramic or other high temperature material that does not adversely react with the pyrolytic reaction. Any handling stems must be compatible with the forming temperatures.

The porous carbon material is treated to either increase water absorption or to make the material hydrophilic. A plasma treatment or a plasma enhanced chemical vapor deposition (PCVD) treatment is believed suitable to add or improve the hydrophilic properties.

One use of the porous carbon particles is use as an absorbent probe that is smaller at a distal end and larger at a fastening end, with its fastening end fastened to a holder and its other, distal end free to contact a fluid to be absorbed, such as blood or other biological fluid. The holder allows easy manipulation of the absorbent probe. The absorbent probe is placed against a blood sample or blood drop(s). Wicking action draws the blood into the absorbent probe. An optional barrier between the absorbent probe and holder may stop blood passing to the holder or wicking to the holder. The porous carbon material is made so that it wicks up substantially the same volume of fluid even when excess fluid is available. The volume of the absorbent probe affects the volume of fluid absorbed.

The absorbent probe is advantageously shaped with an exterior resembling a truncated cone with a narrower and rounded distal end and the wider end is fastened to the holder. Advantageously the holder has a cylindrical post that fits into a recess inside the center of the absorbent probe and extending along the longitudinal axis of the probe and holder. Thus, the truncated conical shape has thick sidewalls that abut the post on the holder, with a distal tip joining the sidewalls and forming the distal end of the probe, with the distal end being flat, rounded or hemispherical.

The holder is preferably, but optionally adapted for use with a pipette because a variety of automated equipment exists to hold and manipulate pipettes. Thus, a tubular holder is preferred, especially one that can fit over the end or tip of a pipette for easy manipulation. A tubular, conical shaped holder is thus preferred, with the absorbent probe on the narrow, tip end of the holder. The wider holder end is open to fit onto a pipette tip. The holder may have outwardly extending flanges located to abut mating structures in holders, drying racks or test equipment to help position the absorbent probe at desired locations in such holders, drying racks and test equipment.

A conical shape of the absorbent probe is believed preferable to help wick the sample quickly and uniformly. Conical probes with flat ends or rounded ends with substantially uniform thickness of the absorbent material are believed preferably. Preferred sampling time is desirably as short as possible with about 2 seconds (less if possible) being most preferred, and up to 15 seconds being acceptable for some medical-related applications. Maintaining the probe in contact with the sample blood droop for between about 2-10 seconds is thus believed sufficient, with a contact time of about 2-5 seconds preferred, and a contact time of about 2 seconds (preferably less) being most preferred, and contact times of 5-10 seconds much less preferred. The contact time is desirably as short as possible. The probe absorbs a predetermined volume of blood during that time, and once saturated does not absorb more blood. The size and shape of the probe can be varied to adjust the volume of absorbed blood and the rate of absorption. Blood volumes of about 7-15 μL are believed suitable, but volumes of about 20 μL and even up to about 30 μL are believed desirable for some applications. Absorbent times will vary for other liquids.

After absorbing a sample, the absorbent probe is then dried, preferably for about 2-3 hours, ideally about 2 hours or less. But the time will vary with the humidity, temperature, volume to be dried and the shape and configuration of the absorbent probe. Drying can be done on a suitable rack or holder, or preferably the absorbent probe and holder can be transferred to a special drying container configured to help drying while minimizing the contact between the probe and the walls of the drying container or other potential contaminant surfaces. As desired, the drying container may have a desiccant to facilitate drying. The drying container may also provide a protective cover or housing which may be sealed for transport to prevent contamination. The cover advantageously has a surface onto which printed indicia may be written to identify the blood sample and provide related information or other information as desired. Advantageously, the preferred dimensions of the container, and the relative positions of the holders within the container, will conform to SBS Microwell plate specifications.

Upon receipt at the location where the testing is to occur, the absorbent probe may be placed in a predetermined volume of liquid solvent by hand or by liquid handling robot to extract the analytes of interest from the dried blood. Physical agitation techniques such as sonication or vortexing of the fluid and/or the absorbent probe can accelerate the extraction analytes of interest from the dried blood into a liquid sample matrix. The fluid is separated from the absorbent probe for further processing (e.g., concentrating), or analysis (e.g., HPLC or GC analysis), while the absorbent probe may be discarded. Physical separation techniques such as centrifugation, evaporation/reconstitution, concentration, precipitation, liquid/liquid extraction, and solid phase extraction can be used to further simplify the sample matrix for further analysis (e.g. HPLC or GC analysis)

There is thus advantageously provided a blood sampling device that includes an absorbent probe made of a hydrophilic, porous carbon material of sufficient size to absorb a maximum of about 20 μl of blood in about 2-5 seconds and having a length of less than about 5 mm (0.2 inches) and a cross-sectional area of less than about 20 mm² and a density of less than about 4 g/cc. The probe is connected to a holder having a manipulating end opposite the probe.

In one embodiment the holder may include a pipette tip or a tapering, tubular structure configured to nest with a pipette tip. Both the probe and holder are made under aseptic conditions, or terminally sterilized. Unsterilized probes are also believed suitable for some applications. The probe may contain dried anti-coagulant, and after use contains dried blood. The holder preferably has a plurality of ribs extending along a length of the holder. The ribs may have a height and length selected to keep the probe from contacting walls of a recess into which the holder and probe are placed for shipment or for extraction of the dried blood in the probe.

The holder preferably has a hollow end opposite the probe and the container may have a first portion with a mounting projection portion sized to fit into and releasably engage the hollow end of the holder. The container preferably has a second portion releasably fastened to the first portion and having a recess configured to enclose a portion of the holder for transportation of the holder. The container advantageously has a plurality of openings allowing air to access the probe. Moreover, the first portion may have a side with an access port therein of sufficient size and located so that indicia may be applied through the port and onto the holder when the holder is on the mounting projection.

Advantageously there are a plurality of holders each with a probe, with each of the plurality of holders having a hollow end opposite its probe. The container likewise has a plurality of elongated mounting projections each sized to fit into and releasably engage one of the hollow ends of the plurality of holders. The second portion of the container has recesses configured to separately enclose each of the plurality of holders in a separate enclosure within the container. Preferably, the plurality of the holders each has a plurality of ribs extending along a length of the holder with the ribs configured to keep the probe from contacting walls of the container. As desired, a desiccant may be placed inside the container to help dry the blood in the probe or keep the blood dried. Each holder may have visible indicia associating the holder with the container and with at least one other holder, such as serial numbers with various portions of the number indicating related holders/probes and the container in which the holders are shipped.

### BRIEF DESCRIPTION OF THE DRAWINGS

These and other advantages and features of the invention will be better appreciated in view of the following drawings and descriptions in which like numbers refer to like parts throughout, and in which:

FIG. 1 shows a prior art blood spot card with an aliquot being applied to the card from a capillary tube;

FIGS. 2a and 2b show an absorbent probe before and after directly contacting a fluid, such as blood, at its source on an animal, such as a human finger;

FIGS. 3a and 3b show an absorbent probe and absorbed sample before and during placement in a container with extraction fluid therein.

FIGS. 4a and 4b show the absorbent probe of FIGS. 3a, 3b before and after the fluid sample is extracted from the absorbent probe;

FIG. 5 is an exploded perspective view of an absorbent probe, a tray to hold a plurality of absorbent probes and a covererable case to hold the tray;

FIGS. 6a and 6b are perspective views of the tray of FIG. 5 inserted into the container with a container lid open and closed;

FIG. 7 is a sectional view of a holder containing an absorbent probe with a protective sheath connected to a hollow holder and covering only the probe and a portion of the adjacent end of the holder;

FIG. 8 is a side view of a holder having outwardly extending ribs for manipulation and a truncated, conical-shaped absorbent probe;

FIG. 9 is a cross sectional view of the probe of FIG. 8;

FIG. 10 is an illustrative view of the holder and probe of FIG. 8 ready to contact and absorb a sample from a subject;

FIG. 11 is an exploded perspective view showing the holder of FIG. 8 in a shipping container having separate compartments for each of a plurality of holders and the probes associated with the holders;

FIG. 12 is a cross sectional view of a portion of a well plate having the holder of FIG. 8 therein, along with an extraction fluid;

FIG. 13a is a bottom elevation view of a further embodiment of a holder having an optional protective sheath thereon;

FIG. 13b is a sectional view of the holder of FIG. 13a, taken along section 13b-13b of FIG. 13d;

FIG. 13c is a top elevation view of the holder of FIG. 13a;

FIG. 13d is a left side elevation view of the holder of FIG. 13c;

FIG. 14a is a top perspective view of a further embodiment of a holder and probe;

FIG. 14b is a top elevation view of the holder of FIG. 14a;

FIG. 14c is a sectional view of the holder and probe of FIG. 14a, taken along section 14c-14c of FIG. 15a is a top perspective view of a container for three holders;

FIG. 15b is a bottom perspective view of the container of FIG. 15a;

FIG. 15c is a side elevation view of the opposing side of the container shown in FIG. 15a;

FIG. 16a is a sectional view of the top portion of the container of FIGS. 15a and 16f, taken along section 16a-16a of FIG. 16b

FIG. 16b is a top elevation view of the top portion of the container of FIGS. 15b and 16f;

FIG. 16c is a side elevation view of the container top of FIGS. 16b and 16f;

FIG. 16d is a bottom elevation view of the container top of FIG. 16f;

FIG. 16e is a side elevation view of the container top of FIGS. 16b and 16f, with the opposing side being a mirror image thereof;

FIG. 16f is a perspective view of the container top of FIG. 15a;

FIG. 17a is a bottom perspective view of the lower portion of the container of FIG. 15b;

FIG. 17b is a top perspective view of the lower container portion of FIG. 17a;

FIG. 17c is a side elevation view of the lower container portion of FIG. 17a;

FIG. 17d is a bottom elevation view of the lower container portion of FIG. 17c;

FIG. 17e is a sectional view of the lower container portion taken along section 17e-17e of FIG. 17d;

FIG. 17f is a top elevation view of the lower container portion of FIG. 17d;

FIG. 17g is a side elevation view of the lower container portion of FIG. 17d, with the opposing side being a mirror image thereof;

FIG. 18a is a sectional view of the container of FIG. 15a with holders therein, taken along 18a-18a of FIG. 18b;

FIG. 18b is a top elevation view of the container of FIG. 18a;

FIG. 18c is a bottom elevation view of the container of FIG. 18a;

FIG. 19 is a perspective view of a case with a plurality of containers and holders therein;

FIG. 20a is a sectional view of a well plate with a holder positioned so its absorbent probe is in extraction fluid;

FIG. 20b is a sectional view of the well plate of FIG. 20a with the holder positioned so its absorbent probe is near to but not in the extraction fluid;

FIG. 21a is a view of an assembled reactor for heating to form an absorbent probe, without the probe material therein;

FIG. 21b is a view of the reactor of FIG. 21a with a portion removed to show the cavities in the reactor;

FIG. 21c is a view of an assembled reactor for heating to form an absorbent probe, with the probe material therein;

FIG. 21d is a view of a probe and stem as formed by the reactor in FIG. 21c;

FIGS. 22a-1 through 22i-3 are sets of three views of illustrative shapes for the porous probe, with each set of views including a top perspective view, a side plan view, and a bottom perspective view;

FIG. 23 is a perspective view of a card having porous probe discs either press-fit into the card or perforated in the card;

FIG. 24a is a front plan view of a further embodiment of a porous probe, with the back plan view being a mirror image thereof;

FIG. 24b is a top plan view of the porous probe of FIG. 24a;

FIG. 24c is a bottom plan view of the porous probe of FIG. 24a;

FIG. 24d is a sectional view of the porous probe of FIG. 24a;

FIG. 25 is a partial view of a holder having the tip of FIG. 24a thereon but shown in broken lines;

FIG. 26a is a front plan view of a further embodiment of a porous probe, with the back plan view being a mirror image thereof;

FIG. 26b is a top plan view of the porous probe of FIG. 26a;

FIG. 26c is a bottom plan view of the porous probe of FIG. 26a;

FIG. 26d is a sectional view of the porous probe of FIG. 26a;

FIG. 27a is a sectional view of a further embodiment of a porous probe;

FIG. 27b is a top plan view of the porous probe of FIG. 27a;

FIG. 27c is a bottom plan view of the porous probe of FIG. 27a;

FIG. 28 is a sectional view of the porous probe of FIG. 24a on a molding core pin;

FIG. 29a-29h is a series views showing a sequence for making a porous probe; and

FIG. 30 is a perspective view of a die for molding a porous probe.

### DETAILED DESCRIPTION

Referring to FIGS. 2-3 and 8, a collection device 10 for collecting various fluids, especially biological fluids and preferably blood, is provided. The device 10 has a sampling end 12 and a holder 14 joined at a juncture 16. The sampling end 12 advantageously comprises an absorbent probe 18 made of a material that wicks up or otherwise absorbs a sample 20 from a fluid source 22, which preferably comprises body fluids and more preferably is blood from a finger-prick or cut 23. The holder 14 may have the absorbent material 18 held in one end, with an opposing end either closed, or preferably open and hollow and optionally configured to allow it to mate with a pipette tip. Releasable adhesives can be used to more securely fasten the parts, but it is believed preferable to force the absorbent probe 18 into a slightly smaller opening in the holder 14 (pipette tip) so the interference fit between the opening and absorbent probe 18 hold the parts together. The device 10 is suitable as a quantitative sampling tool for biological fluids, preferably blood. It is designed for samples to be easily dried, shipped, and then later analyzed.

The juncture 16 is optionally configured to stop wicking of the blood sample 20 past juncture 16, or at least stop wicking adjacent the surface of the absorbent probe 18 at the juncture 16. The absorbent probe 18 thus ends at the juncture 16. The concern is that sample 20 (e.g., blood) will pool inside the holder 14 and not dry out with the sample 20 contained in the remainder of the absorbent probe 18. The juncture 16 preferably comprises a non-porous barrier. It is believed that compressing the outer surface of the absorbent probe 18 at the juncture 16 will restrict wicking by compressing the probe material and thus stop or sufficiently wicking of the fluid sample 20 past the juncture or at least restrict wicking sufficiently to avoid pooling. The juncture 16 could be provided by placing a physical barrier such as wax or plastic between the absorbent probe 18 and the remainder of the sample end 12 and holder 14. The juncture 16 could be formed by joining the absorbent probe 18 to a holder 14 made of material which resists wicking, such as a plastic pipette tip. Various other mechanisms for fastening the absorbent end 18 to the holder 14 will be apparent to one skilled in the art given the present disclosure.

The holder 14 is large enough so a lab technician can manually hold and manipulate the device 10. The holder may take various shapes and is preferably configured to work with tools designed to manipulate pipette tips. By locating the sampling end 12 and its absorbent probe 18 at one end of the holder 14, the user can more easily grip the holder with much less risk of inadvertently touching the blood sample on absorbent probe 18. Further, all portions of the holder 14 can be grabbed by the user or automated equipment, in contrast to the prior art devices which were held by the edges to avoid contamination. Advantageously, the holder 14 is large enough for instruction or cautionary information to be displayed on the holder, such as cautioning the user not to touch the absorbent probe 18.

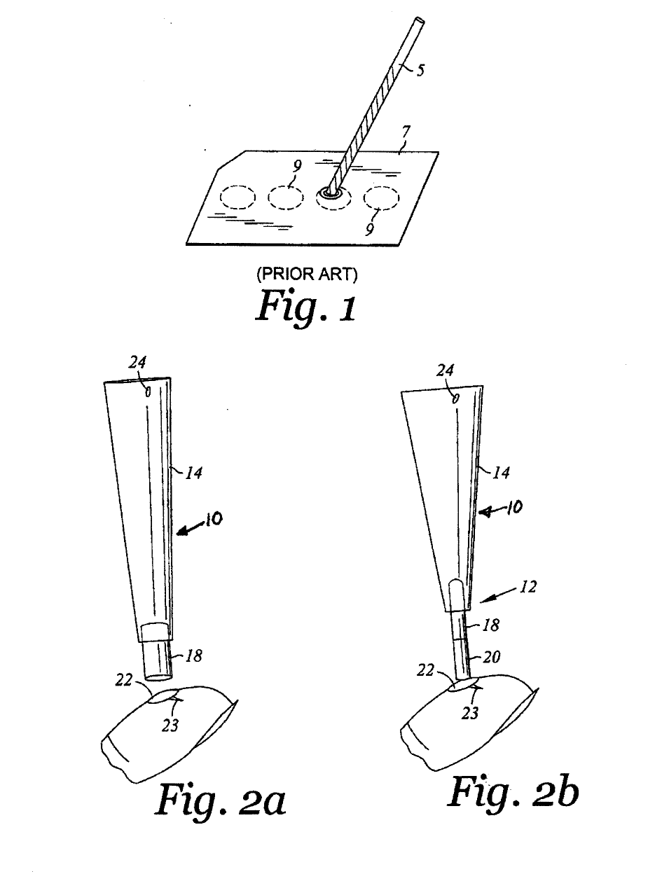

In use, the laboratory technician grabs the holder 14 and places the absorbent material 18 in contact with a fluid source 22 as shown in FIGS. 2a, 2b and 10. The absorbent probe 18 absorbs a fluid sample 20 from the fluid source 22 and wicks the sample it into the absorbent probe 18. The absorbent probe 18 is sized or configured to absorb a predetermined volume of blood before saturation. The absorbent probe 18 has exposed on all sides located outside of the holder 14 so that any exposed surface of the probe 18 may be used to absorb fluid. Excess volumes of sample blood 22 will not be absorbed and will drop off or can be gently shaken off of absorbent probe 18. When the fluid sample 20 is absorbed into sample end 12 then the user preferably places the device 10 in a rack for drying. If a single device 10 is used, the holder 14 can be placed on a book or edge of a table with the sample end 12 suspended in air for drying. If multiple devices 10 are used, a rack with a number of generally horizontal shelves or pairs of posts can be used to hold a plurality of holders horizontally for drying, much like the current racks used with the devices of FIG. 1. Alternatively, well trays exist for holding multiple pipette tips and those could also be used.

The orientation of holders 14 and absorbent probe 18 can be alternated so every other sample end 12 extends from one side of the rack to help avoid contact. Alternatively, the holders 14 could be provided with openings 24 to allow the holders 14 to be hung vertically, with the sample end 12 hanging downward from variously configured hangers. The fluid sample 20 in the absorbent probe 18 is preferably thoroughly dried in order to avoid problems arising from shipping wet biological materials. A drying time of about two hours or more in an ambient, room temperature laboratory environment is believed suitable for a sample volume of about 10-15 μl of blood. Drying times of 2-3 hours are believed suitable. Shorter drying times are desirable, but care must be taken to avoid contamination, as may occur by blowing room air onto the samples to dry them faster. The absorbent probe 18 is positioned so that it does not contact other items or otherwise become contaminated, with special care taken to avoid contamination by materials that could affect the results of the analysis of the sample 20. As desired, the holder and probe 18 may be placed into a container for drying as described later. The probes 18 and container may be placed in a plastic bag along with a desiccant to assist drying and either shipped that way, or shipped after the desiccant is removed.

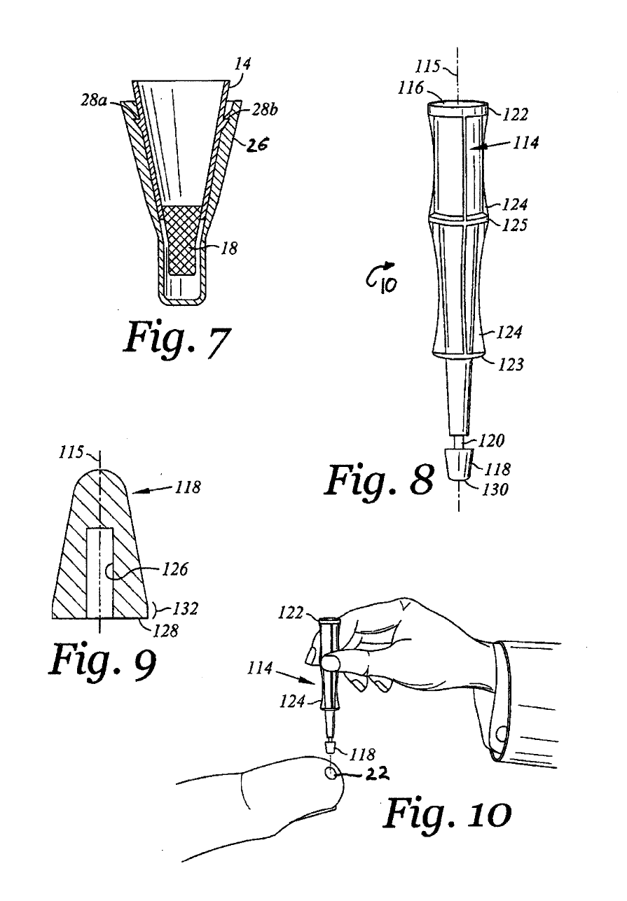

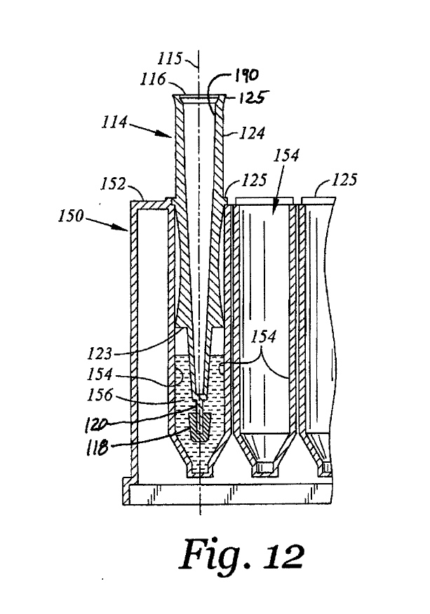

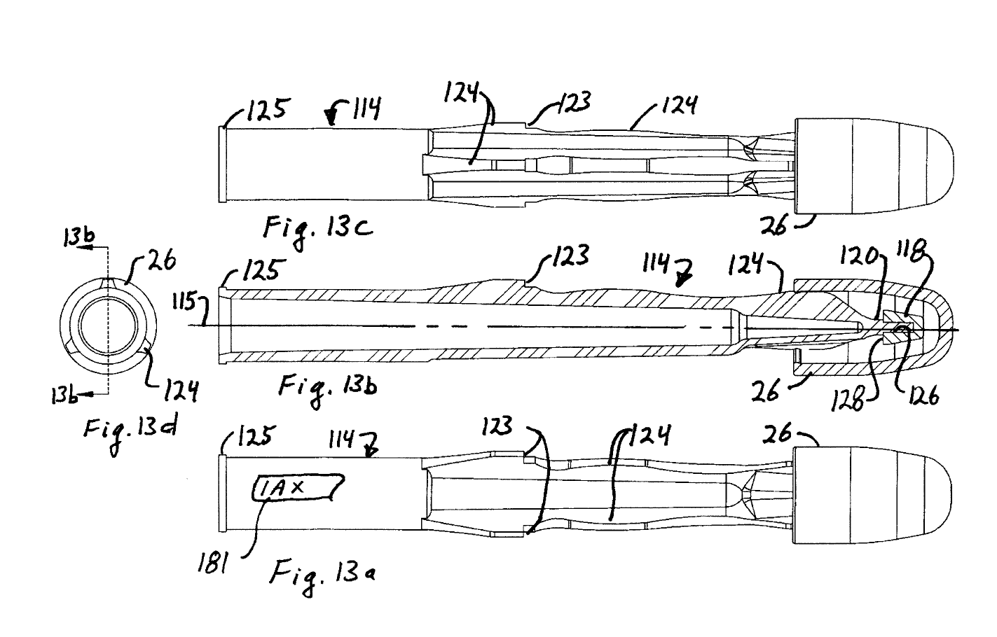

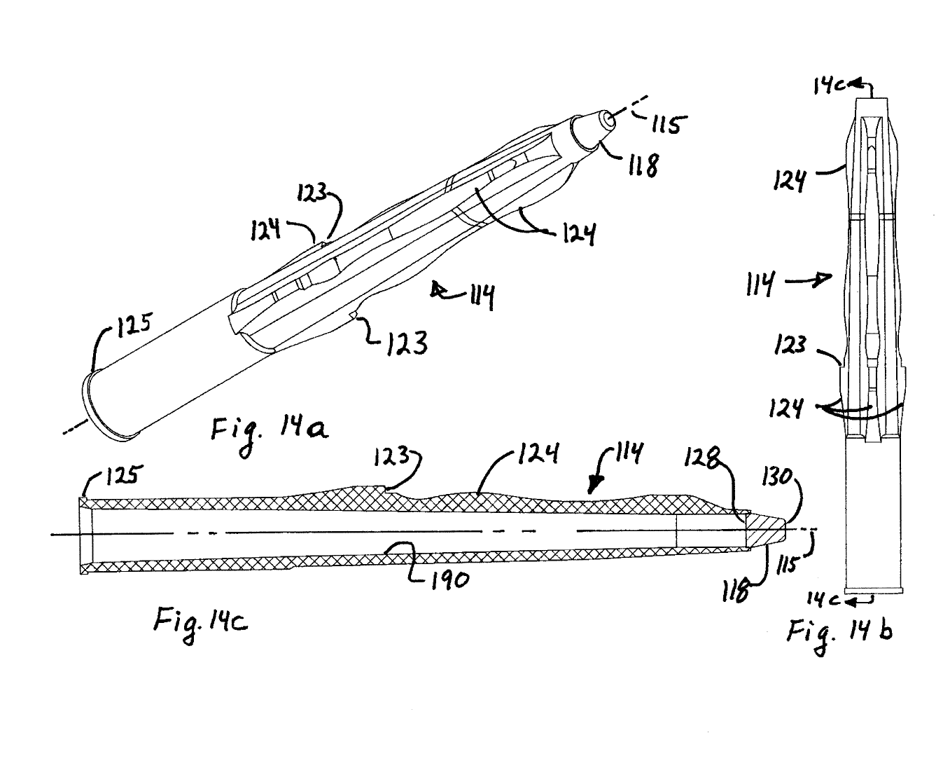

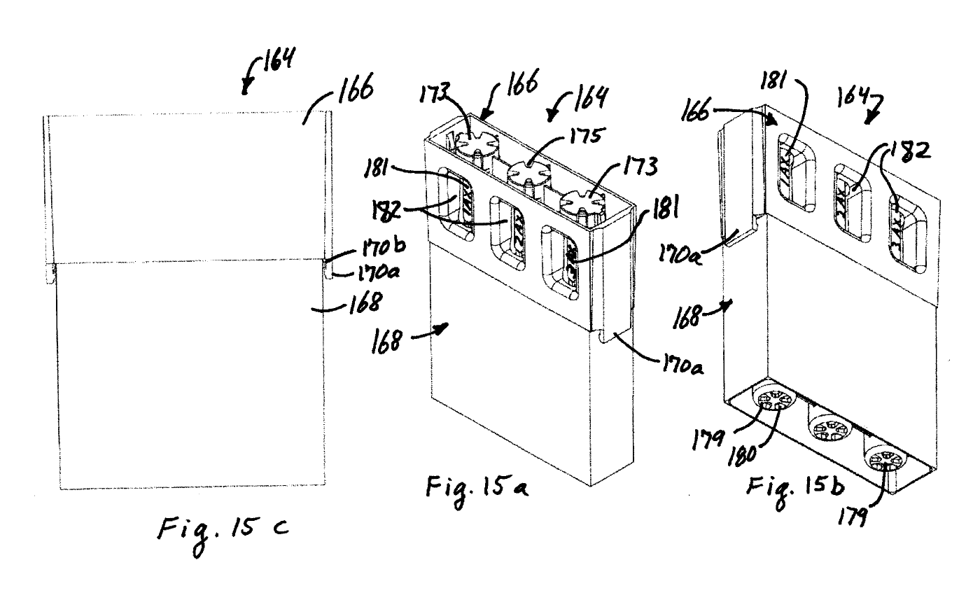

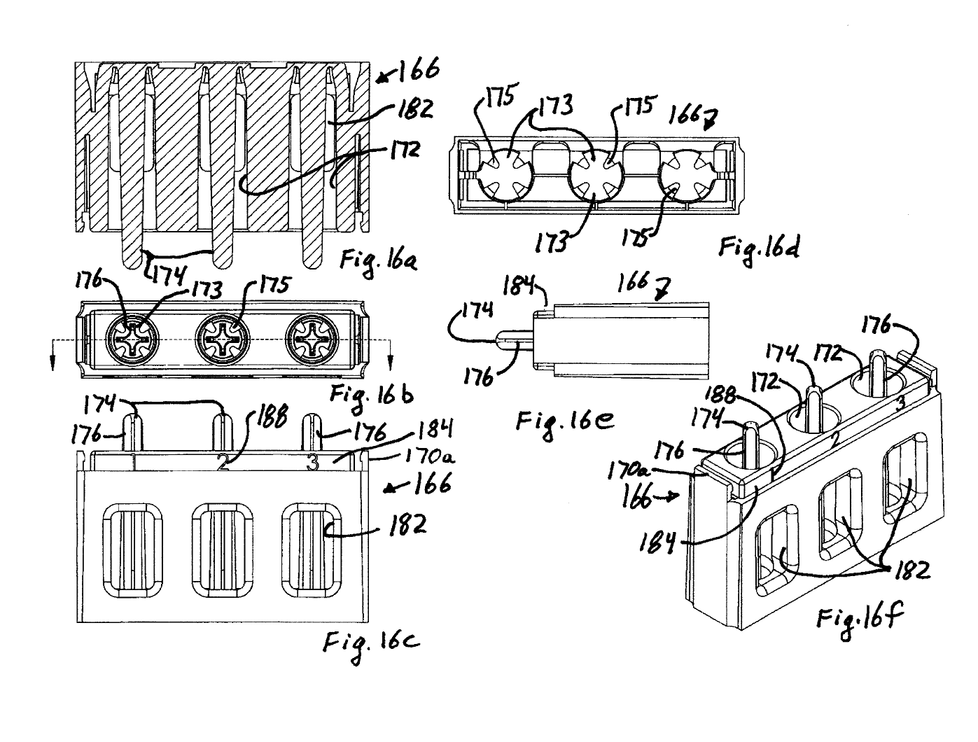

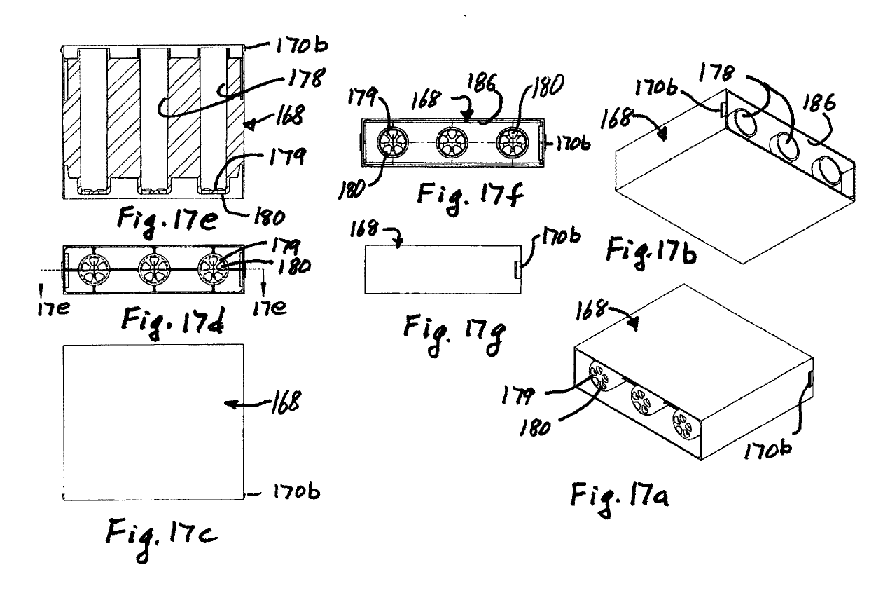

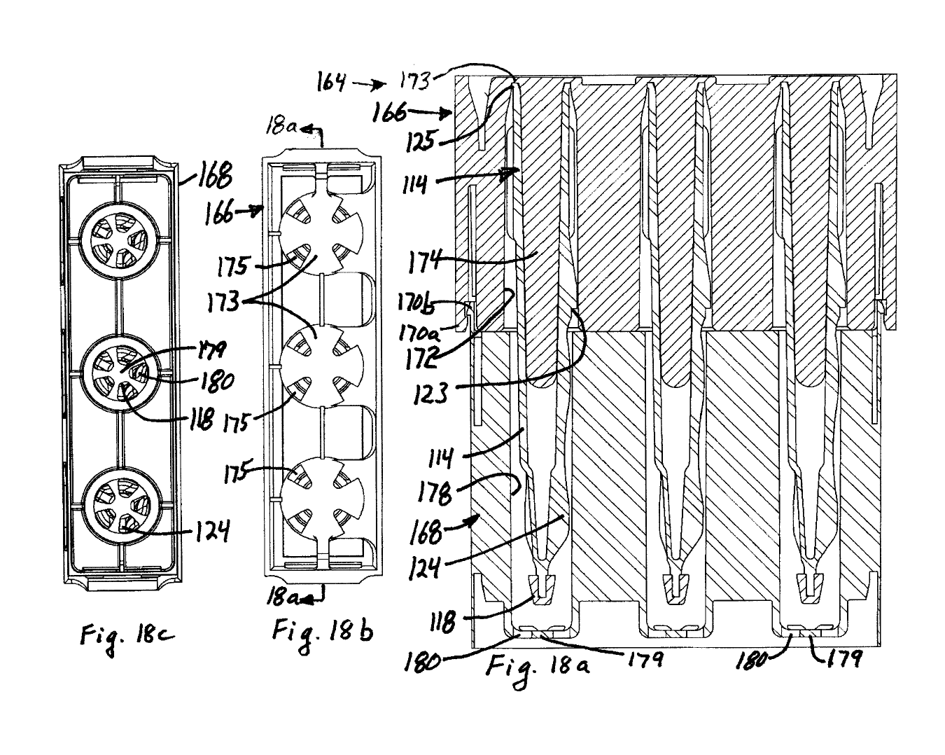

Referring to FIGS. 7 and 13a-13c, an optional protective sheath 26 can be removably placed over the absorbent probe 18 (118) and releasably fastened to holder 14 (114). For example, a tubular sheath 26 with an open end and closed end can have the open end placed over the absorbent probe on sample end 20. An inward facing flange 28a on or adjacent to the open end can releasably engage an outwardly extending flange 28b on the holder 14 to form a snap fit. A threaded connection could also be used instead of the snap fit flanges 28a, 28b. Either configuration works well with holders 14 comprising tubular pipettes or conical pipette tips. Other means of releasably fastening a protective sheath to the holder 18 could be used, including a covered tray configured to hold a plurality of holders 14.

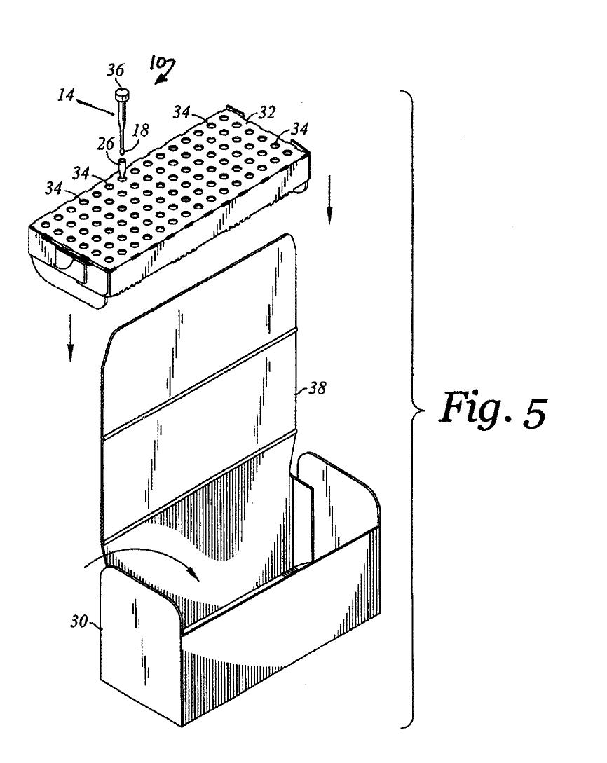

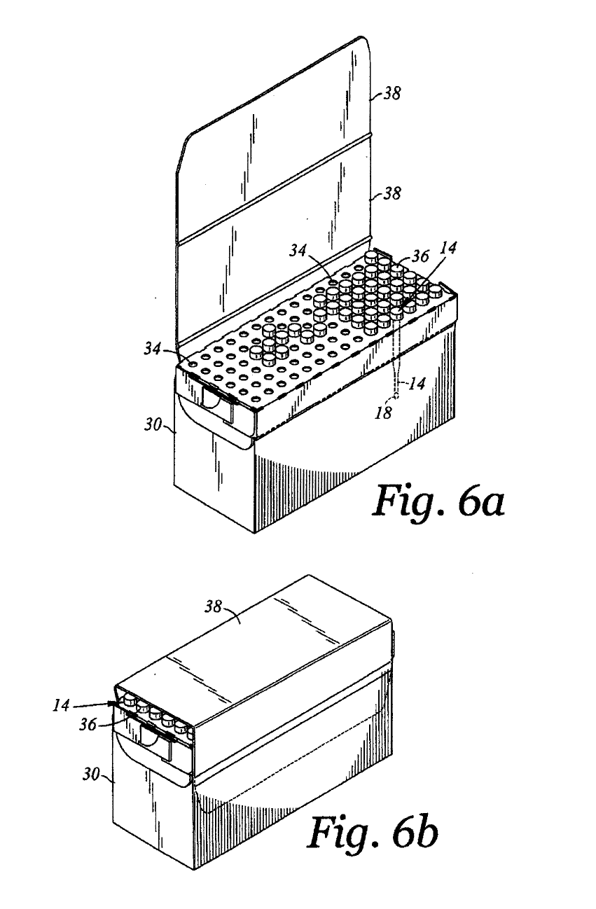

Referring to FIGS. 5-6, the device 10 is preferably contained in a case 30 for transportation. The case 30 may be an expandable container or envelope which is larger than the device 10 and unfolds to allow access to and removal of the device 10 for use, and after the fluid sample 20 is dried allows the device 10 to be placed inside the case 30 which is refolded, sealed and shipped to a laboratory for testing. The container or case 30 and the holders 14 within the container will typically have a human readable label to indicate the container in which each holder belongs. Advantageously, the inside surface of case 30 or the outer surface of case 30 has a writing surface onto which information related to the sample can be placed. Such information could include information for a clinical trial such as code names numbers, barcodes, and RF tags. The name and other information on the subject from which the blood sample is taken, date information, the nature of tests to be conducted, and the project for which the testing is performed. Optionally, a desiccant or moisture absorbing material (not shown) may be placed inside the case 30 for shipping or for storage in order to reduce moisture content and reduce bacteria growth.

Ideally, the case 30 and each device 10 within the case 30 are assigned serial numbers that correspond. Thus, for example, if case 30 contains three devices 10 with blood from a single patient, each device 10 will have a series of common numbers, letters or both indicating they are from the same case and same patient. This labeling helps to associate the device 10 with the appropriate case 30 and its individual holders if they are separated in the laboratory or during analysis. Three devices in a case 30 is believed advantageous since one may be analyzed, one may be used as a backup if there are errors or inconsistencies in initial testing, and one may be used for future verification or retesting, with a series of common numbers, letters etc. making it easier to confirm the devices correspond to the same subject or patient.

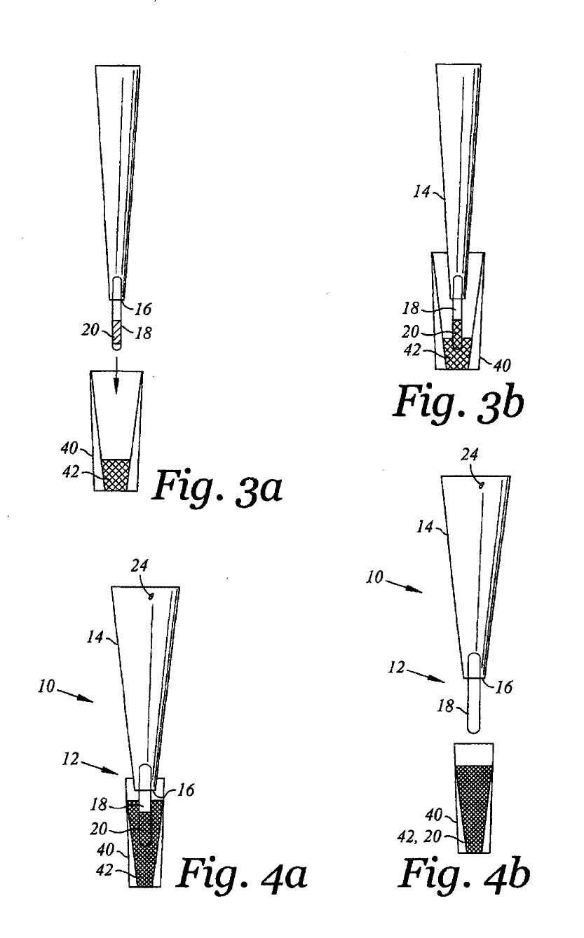

Referring to FIGS. 3a, 3b, 4a, 4b, the devices 10 are usually sent to testing laboratories where, upon receipt, the absorbent probes 18 are tested or analyzed while on the holder 14, or where the probes 18 are removed from holders 14 and reconstituted for testing or analysis. The absorbent probes 18 containing dried samples 20 are placed in containers 40 such as test tubes in which a reconstituting fluid 42 is placed. A plurality of containers 40 may be provided in various racks (FIG. 5) configured to hold the containers. Reconstituting fluid is preferably an extraction fluid or solvent selected to remove the analyte from the dried sorbent tip 18. The fluid 42 varies with the nature of sample 20 and the nature of the test to be performed. The absorbent probes 18 can be removed by various manual and automated means, including pulling the absorbent probe out of the holder with tweezers, or by applying air pressure to the inside of a tubular holder 14. Various means for applying air pressure to a pipette in order to expel the contents of the pipette are also known, and those ways are equally applicable blowing the absorbent probe 18 out of the opening in a pipette tip or other tubular container. The container 40, absorbent probe 18 and its sample 20 are typically agitated to reconstitute the (dried) sample 20 and transfer it to the reconstituting fluid 42 and out of the absorbent probe 18. Sonication or vortexing may be used to agitate the reconstituting fluid 42 and expedite the transfer of the sample 20 from the probe 18 to the fluid 42, with periods of non-agitated soaking used as desired. After the sample 20 is removed from the absorbent probe 18, the probe 18 is removed for the container 40 and may be discarded. The mixture of sample 20 and reconstituting fluid 42 are then available for further processing (such as removing fluid 42 to concentrate sample 20), or further testing (such as HPLC or GC or mass spectrometry analysis).

Alternatively, the holder 14 can be manipulated by a user or by automated equipment so that the sampling end 12 is at or in the open end of container 40 with the absorbent probe 18 positioned in the reconstituting fluid 42. The container 40 and reconstituting fluid can then be agitated, or not, with the holder 14 being used to hold the sample 20 in the fluid 42 until a desired amount of the sample is transferred to the reconstituting fluid 42. The holder and its absorbent probe 18 can then be removed and discarded if insufficient sample 20 remains on the probe 18. The non-sampling end of the holder 14 (opposite the absorbent probe) is preferably dimensionally matched to a pipette tip. The body of the holder is also preferably designed to fit into collection plates for easy extraction, and configured to fit into a rack (pipette tip holding rack or otherwise) for ease of use. The use of pipettes and pipette tips for holders 14 allows automation of the various steps described herein, as the holders 14 can be configured to work with existing pipet or pipetting robotic systems.

Referring to FIGS. 5-6, the absorbent probe 18 is held in a holder 14 comprising a pipette tip. The pipette tip 14 may be placed in a tray 32 adapted to hold a plurality of pipette tip holders 14 and the absorbent probe 18. The sheaths 26 are preferably placed on these pipette tip holders 14 when they are in the tray 32 to further guard against contamination, but that is optional. The holders 14 and sheaths 26 are placed into one of a plurality of holes or openings 34 in the tray 32, which openings are configured to hold the pipette tip holders. The holders 14 may have enlarged ends or removable protective caps 36 to help reduce potential contamination of the inside of the holder 14 and its attached probe 18. The tray 32 may in turn be placed in a shipping case 30, which is shown in these figures as comprising a rectangular box configured to hold the tray, with a foldable lid 38 to cover the caps 36 and secure the pipette tip holders 14 in the shipping case 30. Various other configurations of trays 32 and shipping case 30 can be used.

Referring to FIGS. 2a, 2b and 10, the preferred method places the absorbent probe 18 in contact with fluid, such as blood 22 on a living animal. In its broadest sense living animals include humans as well as other animals. That direct contact with blood while the blood is on the animal eliminates the need to collect blood in capillary tubes and transfer the blood to an absorbent material. Nonetheless, if the fluid 22 is located in a container, capillary tube or any other location, the absorbent probe 18 can be placed in contact with the fluid to transfer a sample 20 (FIG. 2b) of the fluid 22 to the absorbent probe.

Referring further to FIGS. 7-9, the absorbent probes 18 can have various shapes but are preferably circular, rectangular, square or triangular in cross section orthogonal to the longitudinal axis 115. Short cylindrical shapes or frusta-conical shapes are believed preferable for use with pipette holders 14, but the shape may vary to facilitate mounting to and/or removal from holder 14. The absorbent probes 18 are preferably made of a porous carbon material that absorbs a predetermined volume of sample 20 from a larger fluid source 22, regardless of the time the absorbent probe is in contact with the fluid source—at least over a short period of time measured in several seconds. The absorbent probe 18 thus has a dynamic response range measured in seconds rather than fractions of a second. Rods made of a hydrophilic, porous carbon material are believed suitable.

If the porous carbon material is not initially hydrophilic then there are numerous methods for converting the surfaces of the material (both external and internal) into a hydrophilic state, or for improving preexisting hydrophilic properties. Methods for creating or improving hydrophilic surfaces include plasma etching, or PCVD treatments. Treatment with plasma (Corona, Air, Flame, or Chemical) is believed to provide a method of adding polar groups to the surfaces of such materials, including oxygen plasma treatments. Once the porous carbon parts are formed they may also be treated with materials not of pure carbon to improve hydrophilic properties. The use of Tween-40 or Tween-80 to create hydrophilic surfaces is believed suitable. Tween 40 is made of polyoxyethylene (20) sorbitan monopalmilate. Treatment may also occur with other molecules containing both hydrophilic and hydrophobic elements. Likewise, the grafting of hydrophilic polymers to the surface any chemical functionalization of active groups on the carbon particle surface with polar or hydrophilic molecules such as sugars can be used to achieve a hydrophilic surface for probe 18. It is also believed that covalent modification could be used to add polar or hydrophilic functional groups to the surface of probe 18, 118.

U.S. Pat. No. 8,906,448, the complete contents of which are incorporated herein by reference, describes methods of treating material to achieve sufficient hydrophilicity to make hydrophilic articles. The patent describes coating particle surfaces with an oxygenated element and controlling the rate of breakdown of the oxygenated element to leave a corresponding elemental oxide on the surfaces. Illustrative methods include an article, such as probe 18, 118 having carbon or graphite particles with a basal surface, with at least part of the article being porous and then coating at least the basal surfaces of the graphite particles on the porous part with a non-metallic oxygenated element that includes a silicate. The rate of a breakdown of the oxygenated element is controlled to leave a corresponding elemental oxide on the basal surfaces of the graphite particles on the porous part. The breakdown can include decomposition or precipitation. The breakdown can be controlled by using a solvent which may be vacuum impregnated into the porous material. The treated article or probe may be dried, placed in a solution of water and a solvent (such as ethanol and deionized water) and heated, preferably to a temperature over 100° C., and preferably heating to a second temperature of over 300° C., before cooling the material to room temperature. The oxygenated elements selected from oxides of Ti, Al, Si and mixtures of them are believed suitable. There are thus numerous ways of achieving a polar or hydrophilic surface for the probes 18, 118 if the carbonized material and/or absorbent probes 18, 118 are not hydrophilic.

It is believed desirable to form the precursor carbon material for forming porous probes 18, 118 from spherical carbon particles that are agglomerated or formed into a preliminary shape and then carbonizing them to achieve the desired configuration and porosity. Suitable carbon particles are believed to be described in U.S. Pat. No. 7,288,504, the complete contents of which are incorporated herein by reference. This patent describes a process for producing an activated carbon spherule by continuously pre-carbonizing a starting material such as a polymer spherule that is based on styrene and divinylbenzene and comprises chemical groups which form free radicals and cross linkages by thermal decomposition. The chemical groups may be sulfonic acid groups, with the sulfonic acid groups being introduced before and/or during the carbonization step by the addition of SO₃ in the form of oleum. The weight ratio of polymer/oleum 20% being about 1:1. The process includes subsequently discontinuously treating the pre-carbonized polymer spherule in a re-carbonization and activation step to produce the activated carbon spherule. The sulfonic acid groups may be present in the starting material. The starting material is preferably selected from the group consisting of an ion-exchanger, an acid organic catalyst and mixtures thereof. The ion-exchanger is preferably a strongly acid ion exchanger and the acid organic catalyst is preferably a catalyst such as a catalyst for the synthesis of bisphenols or the synthesis of methyl tert-butyl ether (MTBE). The polymer spherule is porous, and preferably macroporous, and/or it may be/or gel-like. The pre-carbonized material is preferably continuously collected in a heat-insulated vessel and is then introduced into a reaction vessel working discontinuously for further pyrolysis (re-carbonization) and subsequent activation. The reaction vessel is preferably a rotary tube.

The activation may be conducted with a mixture of steam and nitrogen at a temperature of from about 850° C. to about 950° C., preferably from about 910° C. to about 930° C., and preferably for a residence time of about 2 to about 5 hours and more preferably for about 2 to about 3 hours. In the process SO₂ may be continuously given off particularly during pre-carbonization and is regenerated, preferably by catalytic oxidation to SO₃, and further processed to sulfuric acid and/or oleum. The pre-carbonizing may be performed in a rotary apparatus working continuously at a temperature from about 100° C. to about 850° C. to form the pre-carbonized polymer spherule. The re-carbonization and activation may be performed in a second rotary apparatus operating at a temperature of from 850° C. to about 950° C. The sulfonic acid groups may be in the form of oleum in mixture with sulfuric acid and in a weight ratio of polymer/oleum 20%/sulfuric acid of about 1:1:05.

The process for producing an activated carbon spherule may also include: continuously pre-carbonizing a starting material which includes a polymer spherule based on styrene and divinylbenzene and comprises chemical groups which form free radicals and cross linkages by thermal decomposition. These chemical groups are sulfonic acid groups that are introduced before and/or during the carbonization step by the addition of SO₃ in the form of oleum in mixture with sulfuric acid, with the weight ratio of polymer/oleum 20%/sulfuric acid being about 1:1:0.5. The process includes subsequently discontinuously treating the pry-carbonized polymer spherule in a re-carbonization and activation step to produce the activated carbon spherule. A similar process continuously pre-carbonizes a starting material of polymer spherule based on styrene and divinylbenzene and includes chemical groups which form free radicals and cross linkages by thermal decomposition. The continuous pre-carbonization may be performed in a rotary apparatus working continuously at a temperature of from about 100° C. to about 850° C. to form a pre-carbonized polymer spherule. The similar process subsequently discontinuously treats the pit-carbonized polymer spherule in a re-carbonization and activation step to produce the activated carbon spherule. The re-carbonization and activation may be performed in a second rotary apparatus operating at a temperature of from about 850° C. to about 950° C. The SO₂ that is continuously given off, especially during pre-carbonization may be regenerated, preferably by catalytic oxidation to SO₃ and further processed to sulfuric acid and/or oleum.

Yet another similar process uses a starting polymer spherule based on styrene and divinylbenzene and includes sulfonic acid groups which form free radicals and cross linkages by thermal decomposition wherein the sulfonic acid groups are introduced in the form of oleum optionally in admixture with sulfuric acid and in a weight ratio of polymer/oleum 20% of about 1:1. The continuous pre-carbonization may be performed in a rotary tube working continuously at a temperature of from 100° C. to 850° C. to form a pre-carbonized polymer spherule. The process may include a re-carbonization and activation step to produce the activated carbon spherule as described above.

The material for the probe 18, 118 is made of a pyrolyzed carbon material involving a chemical reaction forming carbon-to-carbon bonds and creating an open-cell, porous material. The resulting porous carbon micro particles may be formed in a variety of shapes and strengths suitable to a variety of uses.

Pyrolysis refers to heating organic material to cause a chemical reaction in the absence of oxygen. As the temperature of a pyrolysis increases the composition of the remaining carbon material increases as a percentage of the composition's total weight. Therefore, very high temperature pyrolysis is termed carbonization since carbon is the primary material remaining.

However, carbonization is also a thermal degradation that leads to the formation of carbon-carbon bonds, and therefore it also includes or describes a chemical reaction altering the chemical bonds between elements in the composition undergoing pyrolysis and carbonization. A “degree of carbonization” may be used to indicate the percentage of conversion to carbon-to-carbon bonds in a pyrolysis process. Low temperature, long time duration processes may also result in forming carbon-to-carbon bonds and may also be called carbonization.

As used herein, pyrolysis is used to describe the process that includes heating in an inert environment and carbonization is used to describe the reaction when that heating creates carbon-to-carbon bonds and to describe the nature of the final product containing those carbon-to-carbon bonds. The resulting carbonized, porous material used herein advantageously has about 90% or more of the material by weight composed of carbon, and preferably about 92% or more of the material composed of carbon (by weight), and more preferably about 95% or more of the material by weight composed of carbon. Thus, the absorbent probe 18, 118 is about 90% or more carbon, by weight (excluding any stem, etc.). The compressive strength of the material forming the porous tip 18 is preferably about 0.3 to 1 MPa, and more preferably about 0.5 MPa.

Pyrolysis is different from sintering. Sintering is a process involving heat and substantial pressures. Sintering compacts the heated material and results in a more solid, mechanically stable mass. Sintering does not cause a chemical reaction creating the carbon-to-carbon bonds and it does not cause the material to undergo a phase change to a liquid or gas state in the process of forming the sintered material.

A variety of high-carbon content precursors are believed suitable to form the porous carbon micro particles, including cellulose, sugars, polymers, hydrocarbons, etc. Carbonization temperature from about 250° C. to about 1500° C. are believed suitable in an environment designed to deplete non-carbon constituents, which may include oxygen or which may be inert (e.g., nitrogen) if the precursor material has a sufficiently high carbon content and sufficiently low impurity content. A low rate of temperature increase is believed to result in larger retention of the original structure and shape but with lower strength, while a high rate of temperature increase is believed to cause structural collapse and consolidation as the carbon-carbon bonds chemically form and non-carbon materials are burned off in the pyrolytic reaction. The rate of temperature increase or temperature ramp is believed to affect porosity, with lower ramp rates generally increasing porosity but decreasing strength while higher ramp rates generally decrease porosity but increase strength. The pore size of the material is preferably about 10-40 microns so that capillary action draws the fluid into the material and retains it. But the pore size could vary depending on the viscosity of the fluid being absorbed by the material and the size of the particles in the fluid being absorbed, with the pore sizes being at least about 2-3 times larger than the size of the particles to be absorbed. For example, blood cells have a diameter of about 8 microns, so pores larger than about twice that size, about 15-16 microns, are desirable. For biological fluids, pore sizes from about 5-90 μm are believed useful.

The pyrolyzed porous carbon material is advantageously formed in elongated preliminary strips and then cut into a final shape or further formed into a final size or shape suitable for use or for a particular holder. The pyrolyzed porous carbon material may be formed around a handling stem for ease of handling and use. Alternatively, the precursor material may be pyrolyzed in molds having cavities shaped to form the desired shape for the particular use of the material, including shapes formed around a handling stem. Because of the potentially high temperatures the molds may require a metal support with a high-temperature resistant liner made of graphite, ceramic or other high temperature material that does not adversely react with the pyrolytic reaction. Any handling stems must be compatible with the forming temperatures.

The carbonized material generated with a 100° C. ramp is believed to have a mechanical compression strength of 0.5 MPa (similar to a light brick). Carbonized sawdust having a cylindrical or conical or domed shape is believed to be readily achievable. The shape of the probe 18, 118 corresponds to the shape of the cavity in the forming reactor 200 used in its preparation. That shape may be varied as needed.

The carbonized material forming the absorbent tip 18 is not believed likely to react with the absorbed liquid being tested but the reaction will vary with the liquid tested. The carbonized material is not believed to react with blood or other body fluids of humans, other animals, reptiles and avian species. It is believed possible, however, that surface activation by a plasma treatment may cause or increase potential reactivity in which case the surface treatment must either not be used or compensatory steps taken.

The porous carbon material used for probes 18, 118, ultimately results from the carbonization of organic materials, i.e. carbon precursors. In carbonization, the organic material transforms to carbon by thermolysis under an oxygen-free atmosphere to avoid oxidation which may burn and consumes the carbon. Thus, the carbonization advantageously occurs in an inert gas environment such as substantially pure nitrogen, helium, argon etc. But the environment may include carbon dioxide or other gases that do not support a chemical reaction at the maximum carbonization temperature being used and may even oxygen for at least a portion of the carbonization process if it is desirable to burn-off any impurities in the initial precursor material, followed by pyrolysis (without oxygen) to form the probe material.

Various types of carbon precursors can be used to prepare carbon materials and the precursors are advantageously selected to produce carbon that is graphitisable after or upon carbonization. The carbonization used to form the porous probes 18, 118 involves both a physical heating and a chemical reaction by which carbon-hydrogen bonds are converted to carbon-carbon bonds, and by which carbon atoms bonded to other molecules, elements or compounds are converted to carbon-carbon bonds for a very significant portion of the material, advantageously in excess of about 90% of the final material, preferably in excess of about 93% of the material by weight, and more preferably about 95% or more of the final material by weight, and practically to such an extent that any remaining non-carbon impurities do not react with the fluid being tested, especially blood.

Various carbon sources include polymers, aromatic hydrocarbons, pitches, coals, phenolics, alcohols including polyfuryl alcohol, polymers, fats, cellulose (wood), sugars, and hydrocarbons (oils, greases and other petroleum products) in liquid or gas form. In theory, any organic material may be used, with preference given to those materials highest in carbon and containing the fewest non-carbon impurities and further preference given to those materials with impurities that are the most easily disposed of during formation of the porous carbon particles. Polystyrene beads and porous PSDVB (polystyrenedivinylbenzyne) are believed suitable for precursor materials.

Among the carbon precursors with high yield, polymers are believed to be better precursors in terms of ease of processing. Processing high carbon yield hydrocarbons such as pitches and coals may be time consuming and their carbonization may require higher pressures to achieve a high carbon yield. That may be acceptable depending on the configuration of the cavity 202 in the forming reactor 200 and its ability to accommodate pressure. Moreover, these precursors may need some modifications before carbonization in order to achieve the desired porosity. Among polymers, thermosetting polymers don't go through a liquid phase during the carbonization so fine particles of thermosetting polymers, when pressed into a desired shape and placed in a cavity 202 of a reactor 200, are believed suitable for use. Polyfurfuryl alcohol polymers and phenolic polymers are also believed to be suitable for precursors if reduced in size to sufficiently small particles, preferably in the range of 10-100 microns. Polyfurfuryl alcohol is believed to lose less volatiles during curing and carbonization compared to phenolic resins thus achieving a dense final carbonized material and with higher mechanical strength than obtained from phenolic resins.

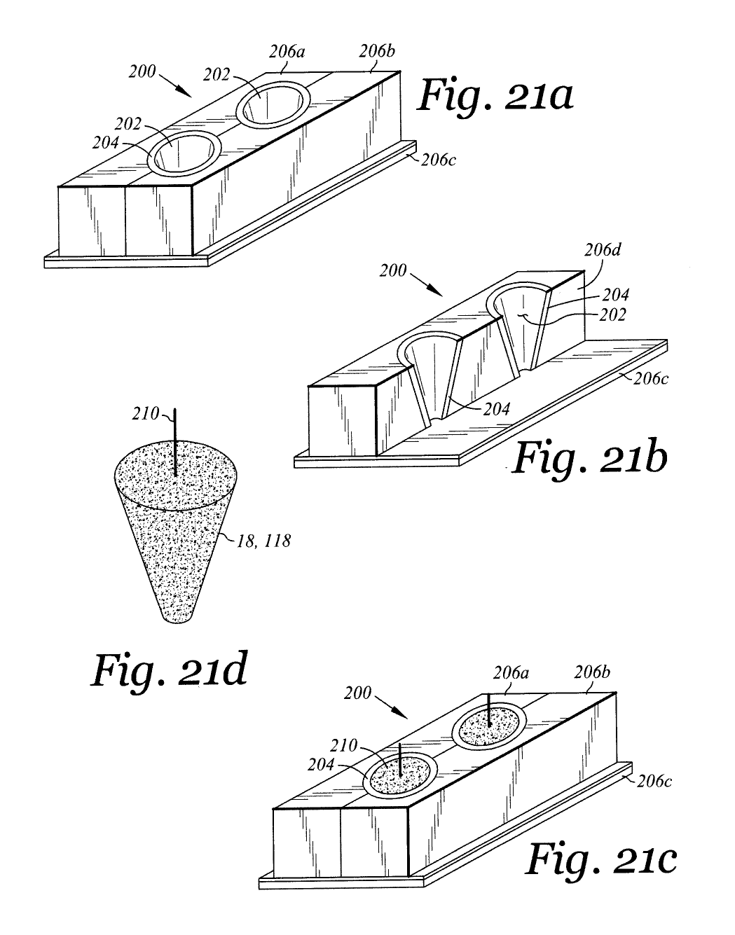

FIGS. 21a-21c illustrate one method of forming the absorbent probe 18, 118 in which a cavity 202 is formed in a forming reactor 200, with one half of the cavity 202 in each of two opposing and abutting sides 206a, 206b of the forming reactor 200. Depending on the temperature used to form the probe 18, 118, a high temperature liner 204 may be used. A split liner 204 is shown but the liner could be a single piece if the cavity 202 is shaped to permit removal of the formed probe 18, 118 from the liner 204. The forming reactor halves 206a, 206b rest on a base 206c which is preferably removable to facilitate removal of the formed probe 18, 118 from the liner and forming reactor. As needed, a high temperature liner may be placed on the top of the base 206c, or may be placed in the bottom of the cavity 202 as part of the liner 204 if the base 206c is not removable. In the illustrated process a handling stem 201 is embedded in the uncarbonized material of FIG. 21c, and remains in the formed probe 18, 118 of FIG. 21d. The handling stem 210 may be used to manipulate the probe 18, 118 after the carbonized material is sufficiently set to allow handling of the probe, and may be used to withdraw the probe from the forming reactor 200. The handling stem 210 may be grabbed manually or by machine or tool to remove and/or otherwise handle and manipulate the probe 18, 118. The forming reactor 200 with the precursor material in the cavities 202 is then heated in an oven or by other mechanisms in an inert environment to carbonize or pyrolyze the precursor material and form the porous carbon probes 18, 118 having an open cell structure.

When present, the handling stem 210 may be fastened to the holder 14, 114 or used to facilitate manufacturing and then removed before the probe 18, 118 is fastened to the holder. Advantageously the handling stem 210 is hydrophobic, and more advantageously is made from a high temperature material suitable for use in and along with the forming reactor 200. Such high temperature materials include glass, ceramics and suitable metals. The handling stem 210 is most easily affixed by embedding it in the precursor material when the absorbent probe 18, 118 is formed. But the handling stem 210 could be affixed after formation of the absorbent probe, as by forming a hole in the probe to receive the stem and then inserting the stem into the hole and fastening it there. The hole could be formed by a removable plug used during formation of the probe 18, 118, or by drilling or by displacing material. The handling stem 210 could be press-fit into the hole, threaded and screwed into the hole, or affixed by suitable adhesives to the hole or to other parts of the probe.

Thus, a forming block 200 having at least one and preferably a plurality of cavities 202 therein is filled with precursor material having a high carbon content of over 90% carbon and preferably over 95% carbon and more preferably over 98-99% carbon. The boundaries of the cavities 202 may be lined with a high temperature liner depending on the temperature used to form the material. A handling stem 210 may be positioned in the carbon precursor material and, as needed, held in place by fixtures. The forming block 200 is then heated to a desired temperature for a desired time in an inert environment in order to carbonize or pyrolyze the precursor material and form carbon-to-carbon bonds in an open cell structure to create the porous probe 18, 118.

It is also believed that porous carbons may also be achieved by foaming. Carbon foams can be produced from different precursors of polymers and pitches. Carbon foams from polymer precursors can be prepared by polymerization of a carbon resin combined with foaming agents followed by carbonization. Typically, polyurethane and phenolic resins may be used as foaming agents. The resulting carbon foam is a reticulated vitreous carbon (RVC). Important process variables in carbonization of a carbon resin include the concentration of the resin, the type of solvent, the solution viscosity and the carbonization temperature and rate. The foam is typically carbonized at 700-1100° C., however. During the production of RVC, a linear shrinkage of approximately 30% may occur. Vitreous carbon foams may be produced in several pore sizes. A vitreous carbon foam with 97% pore volume is believed achievable with a low density and relatively even pore distribution and moderate mechanical strength of 0.07-3.4 MPa. If the foaming method uses pitch precursors no foaming agent or stabilization step is believed necessary. But the foaming process requires pressure to control the evolving gases which makes the process more complicated compared with the method using polymer precursors.

One carbon precursor derived from carbon foam is described in U.S. Pat. No. 8,372,510, the complete contents of which is incorporated herein by reference.

Carbon foams may be prepared by several routes. Highly graphitizable foams may be produced by thermal treatment of mesophase pitches under high pressure. Heating mesophase pitch while subjected it to a pressure of 1000 psi to produce an open-cell foam containing interconnected pores with a size range of 90-200 microns is believed to be known. After heat treatment to 2800° C. the solid portion of the foam develops into a highly crystalline graphitic structure with an interlayer spacing of 0.366 nm and the resulting foam is believed to have a compressive strength greater than 3.4 MPa or 500 psi for a density of 0.53 gm/cc.

U.S. Pat. No. 6,776,936, the complete contents of which are incorporated by reference, forms carbon foams with densities ranging from 0.678-1.5 gm/cc by heating pitch in a mold at pressures up to 800 psi. The foam is believed to be highly graphitizable. Porous carbon is also believed producible from mesophase pitch followed by oxidative thermosetting and carbonization to 900° C. with the foam having an open cell structure of interconnected pores with varying shapes and with pore diameters ranging from 39 to greater than 480 microns or larger. Such porous graphite particles may be prepared by introducing pitch into a mold where the pitch has a characteristic boiling point at a given pressure and for a given temperature. The air is purged from the mold and the pitch is pressurized between a preselected initial processing pressure and a relatively lower final processing pressure. The preselected initial pressure serves to increase the boiling point of the pitch above the boiling point at the final processing pressure. The pitch is heated while at the initial processing pressure to a temperature below the solidification point but above the boiling point which typically occurs at the final processing pressure. After heating the pitch is depressurized from the initial processing pressure to the final processing pressure while maintaining the process temperature above the typical boiling temperature at the final pressure to produce a porous artifact. The porous artifact is heated to a temperature that solidifies and cokes the porous artifact to form a solid, porous carbon, which is cooled to room temperature. A simultaneous release of pressure may heat the solid, porous carbon to a temperature between 900° C. to about 1100° C. to completely carbonize the artifact. The process may further include heating the solid porous carbon artifact to a temperature between 2500° C. to about 3200° C. to graphitize the artifact thus producing a porous graphite artifact. The processed pitch may be mesophase, isotropic, synthetic, coal=based, petroleum based or mixtures thereof.

Carbon foams that are not highly graphitizable may be prepared from coal-based precursors by heat treatment under high pressure to give materials with densities of 0.35-0.45 g/cc with compressive strengths of from 800 psi at a density of 0.27 g/cc to a strength of 2000-3000 psi (thus a strength/density ratio of about 6000 psi/g/cc). These foams have an open-celled structure of interconnected pores with pore sizes ranging up to 1000 microns.

U.S. Pat. No. 5,888,469, the complete contents of which are incorporated herein by reference, describes production of carbon foam by pressure heat treatment of a hydrotreated coal extract. The carbon foam is anisotropic and may be formed by hydrogenating and de-ashing bituminous coal and then converting that hydrogenated bituminous coal into asphaltenes and oils in a solvent (e.g., tetrahydrofuran). The asphaltenes are separated from the oils, preferably in an inert gas. Then the asphaltenes are coked by heating at a temperature of about 325° C. degree to about 500° C. for about 10 minutes to 8 hours to devolatize and foam the asphaltenes at a pressure of about 15 to 15,000 psig, after which the carbon foam is cooled before being graphitized at a temperature of at least about 2600° C. After the coking but before the graphitizing, the carbon foam is calcinated. The process is believed to create carbon foam with voids of generally uniform size, whereby the bituminous coal is converted into an anisotropic, calcined, graphitized, carbon foam having voids of generally uniform size. These resulting materials are believed to have high compressive strengths of 600 psi for densities of 0.2-0.4 gm/cc (strength/density ratio of from 1500-3000 psi/g/cc). These foams are believed to be stronger than those having a glassy carbon or vitreous nature which is not graphitizable.

Carbon foams can also be produced by direct carbonization of polymers or polymer precursor blends as described in U.S. Pat. No. 3,302,999, the complete contents of which are incorporated herein by referenced. The carbon foams by heating a polyurethane polymer foam at 200-255° C. in air followed by carbonization in an inert atmosphere at 900° C., preferably without cooling between the lower and higher temperature steps. These foams have densities of 0.085-0.387 g/cc and compressive strengths of 130 to 2040 psi (ratio of strength/density of 1529-5271 psi/g/cc).

Carbon foams may also be used to create porous carbon particles as described in U.S. Pat. No. 5,945,084, the complete contents of which are incorporated herein by reference. This patent prepares open-celled carbon foams by heat treating organic gels derived from hydroxylated benzenes and aldehydes (phenolic resin precursors). The foams have densities of 0.3-0.9 g/cc and are composed of small mesopores with a size range of 2 to 50 nm. Carbon foams are also believed suitable to create porous carbon particles by pyrolysis of phenolic resins, resulting in foams with a density range of 0.1-0.4 gm/cc, with compressive strength to density ratios from 2380-6611 psi/g/cc. The phenolic resins may result in ellipsoidal shaped pores shape with pore diameters of 25-75 microns using a carbon foam with a density of 0.25 gm/cc.

The shape of the pores may also be varied as discussed in U.S. Pat. No. 6,103,149 to Stankiewicz, the complete contents of which are incorporated herein by reference. That patent prepares carbon foams with a controlled aspect ratio of 0.6-1.2, for uses where a completely isotropic foam are desired with an aspect ratio of 1.0 being preferred. An open-celled carbon foam is produced by impregnation of a polyurethane foam with a carbonizing resin followed by thermal curing and carbonization. The pore aspect ratio of the original polyurethane foam is thus changed from 1.3-1.4 to 0.6-1.2.

While the various methods of manufacturing the porous carbon probe 18, 118 each have advantages and disadvantages, the use of carbon in the probes 18 is believed especially advantageous because of their inertness relative to bodily fluids and their compatibility with bodily fluids—which are carbon based. Illustrative bodily fluids usable with the probes 18, 118 include blood, blood plasma, tears, urine, synovial fluid and saliva so the probes will contain those fluids in the liquid state, and I the dried state. Other liquids believed suitable for use with the probes include slurries from homogenization processes, with the dried form also being contained in the probe. The porous probes are believed suitable for use with water and samples containing about 40% or more water, with the dried probes containing the samples after the water are dried. Liquids other than water may be absorbed, with the porosity, pore size and probe size and probe shape being varied to achieve various combinations of absorption volume and absorption times.

Porous carbon probes are believed to be useful to provide a variety of porosities. Porous carbon probes are believed to allow a wider range of surface modifications that conventional, polymer based porous materials. Moreover, polymer based porous materials have inherent organic materials in them which may leach out, especially in the presence of strong organic liquids, degrading the liquid sample 22 or testing of that sample. The porous carbon probe is believed to provide more control over the shape of the pore and size of the pores than polymer absorbent materials. Further, porous carbon probes are not believed to swell as much as polymer based porous material. Additionally, because the probes are made of carbon, most organic materials can be used as a source of carbon from which the porous carbon probes may be made.

The material of probe 18 must be porous in order to absorb fluid. The internal volume of the absorbent probe material (pore volume) is preferred to be between about 30% and 50% of the total volume of the material, and preferably 40% or greater. Additionally, the nature of the absorption requires small pores (preferably cylindrical tubes although irregular shapes are also sufficient) that are nominally 20-50 micron in diameter or largest cross-sectional dimension. The desired pore size will vary with the liquid sample 22. The shape of the probes 18, 118 preferably is such that concave surfaces are avoided because the concave surface may encourage fluid sample 20 to “hang on” through surface tension to the outer surface of the probe, rather than be absorbed inside the probe and that may alter the desired volume of liquid absorbed by the probe. The extent of variation caused by this “hang on” will depend in large part on the surface tension of the particular liquid sample 20.

The density of the porous, pyrolyzed carbon varies with the pore volume, which is advantageously about 40% or more of the volume of the absorbent probe 18, 118, excluding any stem or holder embedded in the probe. The bulk density of the absorbent probe material is believed to be about 0.05 to about 0.8 grams per cubic ml. As the density increases the time to absorb fluid sample 20 increases. As the porosity increases, the time to absorb a fluid sample 20 decreases—as long as the pore size is small enough to create capillary action for a given viscosity of the sample 20. For blood absorption a shorter time is believed preferable when the sample 40 is taken from a live subject providing a live source of fluid 22, as by contacting the probe 18 with a cut 23 in a person's finger. Absorption times of about one or two seconds are believed suitable for blood from a live subject. The times will vary with the volume of fluid sample 20 desired and its source fluid 22. The density affects the time for the dried fluid sample 20 to be reconstituted. Blood absorbed by the lower density material reconstitutes faster than does the higher density material.

As the contacting area of the hydrophilic probe 18 increases the time to absorb the fluid sample 20 decreases. Thus, for faster absorption larger contacting areas are used on the absorbent probe 18. But a larger area on probe 18 does not maximize the absorption rate if the area of the fluid source 18 is much smaller than the contacting area of the absorbent probe 18. Thus, the anticipated size of the source 18 is advantageously considered in configuring the absorbent probe 18. A cylindrical probe 18 with a diameter (or other shape having a size providing an equivalent area) of about 2-6 mm is believed suitable for use with blood, with diameters of about 3-4 mm being preferred for samples of about 10-14 mg of blood, absorbed in about two seconds, for the most preferred probes 18, 118. A probe length of about 1-5 mm is believed suitable when the sample 20 and fluid 22 are blood, with lengths of about 2-3 mm being preferred. Areas of about 6-20 mm² are believed especially suitable for the tip of the absorbent probe 18 when the fluid source 22 comprises blood formed by a finger prick, with areas of about 10 mm² being believed even more suitable. Shapes that maximize surface area of the contacting portion of the absorbent probe 18 while reducing dripping are believed desirable. Flat ended cylinders or semicircular ends on cylindrical probes 18 are believed desirable, but various configurations can be used.

The volume of the absorbent probe 18 is selected to absorb a predetermined volume of sample 20 from source 22. When the sample 20 is blood, a sample volume of about 18-21 μL is believed suitable, and absorbent probes 18 about 3 mm-3.5 mm in diameter and about 2 mm long, with a density of about 0.1 to 1.3 g/cc are believed suitable for absorbing that volume of blood in about two seconds. Similarly, probes 18 about 4 mm long absorbing a volume of about 8-12 μl, and preferably about 10 μl, in 2-4 seconds are believed desirable. For these probes, it takes about two hours at ambient room temperature to dry the sample 20 absorbed into the absorbent probe 18.

The device 10 is manufactured under sterile or aseptic conditions in accordance with international safety standards for direct subject sampling. Alternatively, the device 10 may be terminally sterilized after manufacture and before packaging. The device 10 is preferably a single use device to be discarded after the absorbent probe 18 is used once.

While the sample 20 is preferably dried, the absorbent probe 18 may be covered by suitable protective sheath 26 (FIGS. 7, 13) or placed in a sealed container so the device can be transported to a location for analysis. Shipping wet biological fluids requires special steps, but it can be done.

Referring to FIGS. 8, 13 and 14, various configurations of holders 114 are shown. In FIGS. 8 and 13, the holder 114 has a projection onto which the probe 118 is mounted and in FIG. 14, holder 114 has a tubular holder with an opening into which the probe 118 fits. Except for the way the absorbent probe 118 is held the holders are generally the same. Referring first to FIGS. 8 and 13, the holder 114 extends along a longitudinal axis 115, having a larger diameter open end 116 sized to fit over and nest with a pipette tip, and a smaller diameter tip that is closed. Advantageously, the tip has a post 120 extending therefrom. This embodiment has no circular flanges extending perpendicular to the longitudinal axis 115. The flange 123 (FIG. 8) adjacent probe 118 is preferably omitted in this embodiment to avoid retaining any fluid on the flange during extraction. A plurality of longitudinal ribs 124 extend along a portion of the holder length and preferably extend between adjacent flanges. Advantageously 3-4 ribs 124 are used, equally spaced around the outside of the holder 114, to allow easy gripping and manipulation by a person's fingers. The ribs extend along the length of the holder along axis 115, between the manipulating end and the probe end of the holder. In the illustrated embodiment of FIG. 8, the ribs 124 curve inward toward the holder 114 between the flanges 124 to better conform to the tips of a person's fingers, while the ribs 124 in FIG. 1 have a different curvature. A holder about 2-4 inches long, with flanges 122 spaced about every one or two inches is believed suitable. The ribs 124 may serve several functions in addition to making it easier to grip and manipulate the device 10. The ribs 124 may help align the device 10 with the portions of case 30 configured to receive each device 10. The case 30 may have recesses configured to receive one or more ribs 124, or an opening in the case may have recesses configured to receive one or more ribs and guide the rib into position within the case. The ribs 124 may also hold or position probe 18, 118 in spaced relation to the adjacent wall of case 30. The ribs 124 may also be configured to allow a robotic handler grab and position the device 10 and its associated probe 18, 118.

Referring to FIGS. 8-9, the post is advantageously cylindrical in shape and extends along longitudinal axis 115. The absorbent probe 118 has a cavity 126 shaped to conform to the post 120, and preferably slightly smaller so the probe 118 resiliently grips the post 120 to hold the probe on the post. An optional adhesive could be used as desired to further hold the probe and post together. The probe resembles a truncated cone with a wider diameter base 128 and a narrower diameter distal end 130 that is preferably rounded. The base end 128 may have a cylindrical section 132 of uniform diameter before tapering toward the distal end 130. The cavity 126 extends about ⅔ the length of absorbent probe 118 measured along the axis 115. The interior end of cavity 126 forms thick sidewalls on the absorbent probe 118. The sidewall thickness increases toward the base 132 and the distance from the end of cavity 126 to the outermost portion of distal end 130 along axis 115 is preferably two or more times greater than the thickness of the sidewalls. Advantageously, the probe 118 is configured so the distal end 130 rapidly absorbs blood, and rapidly wicks the blood throughout the body of the absorbent probe 118.

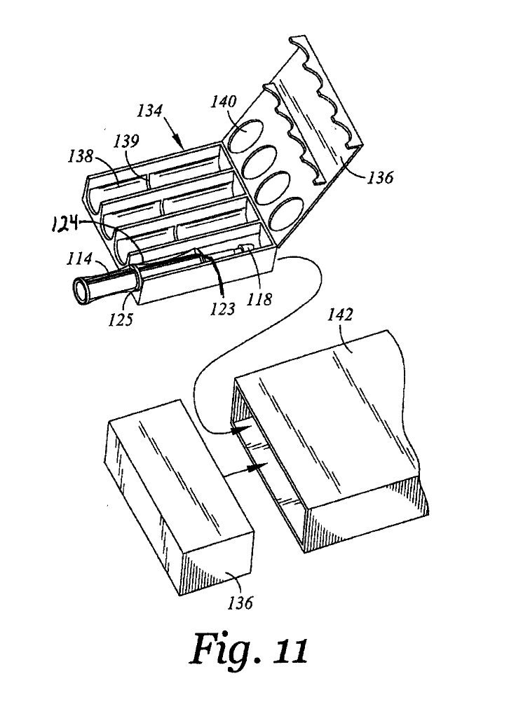

The absorbent probe 118 is made of porous carbon material with a controlled porous volume. An internal standard may optionally be pre-adsorbed onto the probe 118 and dried. The probe 118 and holder 114 are placed in sterile or aseptic packaging and provided to the user in single units or packages of plural units, such as four as shown in FIG. 11, or three shown and described later.

Referring to FIGS. 2 and 10, the holder 114 is held by hand and a user places the absorbent probe 118 in contact with blood, as for example, arising from a finger prick. The probe 118 could be placed in contact with the blood various ways, including immersing in a sample in a container, swabbing a cut, contact with a pool of blood, or other means. The blood is absorbed by the probe 118 in the timelines discussed herein. Advantageously, the probe 118 is sized and configured to absorb a predetermined volume of blood in a predetermined amount of time, such as 10-15 ul in about 1-4 seconds and preferably less.

Referring to FIG. 11, the holder 114 may be placed in a container 134 having removable lid 136 and one or more racks or compartments 138 or racks configured to receive one or more holders 114. Advantageously, the compartments 138 may comprise tubular compartments, preferably cylindrical compartments, having an inner diameter slightly larger than the diameter of flange 122 and slightly smaller than flange 123 so flange 123 abuts a wall on the container 134 to limit the distance the holder 114 is inserted into container 134. The flange 123 and adjacent walls of tubular compartment 138 help restrain the holder 114 from moving laterally. Air holes 140 in the walls of container 134 may be provided to allow air to circulate through the container 134 at the location of the absorbent probe 118 and are preferably large enough to sufficient to dry the probe in 2-3 hours in an ambient laboratory room temperature and humidity. The illustrated embodiment places circular holes in opposing walls of the container 134 located at the absorbent probe 118 so air can pass through the container at the location of the probes to dry them. A bottom portion 142 of the container may fit on the upper portion 136 to cover the holes for shipping. Lid 134 is placed on the top of the container 134 and configured to provide a wall close to or abutting the flange 125 so the holders 114 don't move much during shipping. Instead of or in addition to the flange 125, the ribs 124 may extend along a sufficient length of the holder 114 and fit close enough to the walls of the recess 154 in the well plate or container 150 (FIG. 12) so as to position the probe 18, 118 relative to the recess in which the probe is placed. As desired, a foam material or other resilient material may be provided to abut portions of the holder 114 and hold it in position during shipping. A surface on container 134, lid 136 or bottom 142 is preferably provided for adding information on the holders 114 and probes 118, such as the name or identification number of the person associated with the blood on a particular probe 118. Thus, a user can grab the end of a holder 114, absorb a blood sample on probe 118, place the holder and absorbed sample in container 134 and allow the sample to dry. When dried, the lid and bottom can be put on the container 134 for shipping.

Referring to FIGS. 15-18, a further transporting or shipping container 164 is shown having a removable top 166 and bottom 168 portions releasably held together by an optional snap lock 170a, 170b on at least one and preferably on two opposing sides of the container 164. The top 166 could be hinged, but separable parts are preferred. The top 166 is preferably rectangular in cross-section with an exterior top that is flat and may rest securely on a flat surface such as a table during use. The top 166 has a plurality of recesses 172 (FIG. 16), preferably cylindrical, configured to receive the end of holder 114 during use of the container and having an end wall 173. Three recesses 172 are preferred, but the number can vary. A mounting projection 174 extends from the center of each recess 172. Each mounting projection 174 advantageously has a number of ribs 176 extending outward from the projection and along a length of the projection. A hole 175 extends through the end wall 173 between each rib so air can circulate through the holes or openings 175. Advantageously there are several air-flow openings 175. As best seen in FIG. 16a, the bottom of the circular recess 172 is slightly conical so it inclines slightly inward toward the mounting projection 174 and the mounting projections are slightly conical so the bottom of the mounting projection 174 tapers slightly tapered outward. The manipulating end of holder 114 with the (optional) flange 125 fits between these two tapered portions.

As best seen in FIG. 18a, the manipulating end of holder 114 adjacent the flange 125 (FIG. 13) is hollow and that end and the ribs 176 are sized to nest together so the holder 114 fits over the mounting projection 174 with a slight interference fit. Advantageously, the holder 114 has a slightly tapered, internal, conical passage that mates with a slightly conical exterior shape on mounting projection 174 and its ribs 176 so the two parts wedge together with the ribs 176 abutting the inside of the manipulating end of holder 114. If desired, the outer diameter of the end of the holder 114 may abut the walls of the recess 172 at the tapered bottom of that recess in order to form a slight interference fit, but that is not believed necessary.

The holder 114 and container parts 166, 168 are preferably made of molded plastic and given the molding tolerances sight interference fits between the holder 114 and one or both of the mounting projection 174 or recess 172 are possible. The end wall 173 may abut the end of the holder 114 or the end flange 125 on the holder 114 to limit the maximum relative motion between the mounting projection 174 and the manipulating end of holder 114. The length of projection 174 is long enough to ensure alignment of the holder 114 releasably fastened to that projection. The projections 174 are parallel, and coincide with longitudinal axis 115 of the holders and the axis of a recess 173 in the bottom 168 during shipment or transportation of the holders.

Referring to FIGS. 15, 17 and 18, the bottom portion 168 of the container 164 has recesses 178 (FIG. 17e, 17b), each with a bottom 179. The recesses 178 are located to match with the recesses 172 in the top 166 to form compartments within which the holders 114 and probes 118 are releasably held for transportation. The recesses 172, 178 in the top and bottom portions 166, 168, respectively, are preferably cylindrical recesses to form cylindrical compartments. The bottom 179 has air openings 180. Five openings 180 are shown but the number may vary. As best seen in FIG. 118a, the holder 114 has ribs 124 sized to fit inside the recesses 178, preferably with a small clearance between the outermost portion of ribs 124 and the adjacent walls forming cylindrical recesses 178. The ribs 128 and recesses 178 cooperate to keep the probe 18, 118 from hitting the walls forming the recesses 178.

The end of the holder 114 adjacent flange 125, or the flange 125 on the manipulating end of holder 114 abuts the closed end 173 of the wall forming the recess 178 to limit movement of the holder 114 relative to the recess 172 and top portion 166 of the container 164. That occurs when the holder 114 is wedged onto the mounting projection 174 with a slight interference fit. The holders may be pushed off of the mounting projection 174 by inserting prongs or fingers through openings 175 in the bottoms 173 of recesses 172. If the holders 114 are not wedged onto projections 174 then if the top 166 is vertically above the bottom 168 the holders 114 will fall toward the end 179 of the recess 178. The notches 123 in the ribs 124 or a similarly located flange or other projection on holder 114 will abut the open edge forming recess 178 to limit the relative position of the holder and its probe 118 within the recess 178. Thus, the tubular compartment formed by aligned cylindrical recesses 172, 178 contain the holder 114 and its associated probe 118, with the shape of the holder 114 and mounting projection 174 limiting movement within the top portion 166 of the container 164, and with the notch 123 on the holder 114 and the ribs 124 limiting movement within the bottom portion 168 of the container.

In use, the lid or top 166 of the container 164 has a holder 114 inserted into each recess 172 of the top 166 and preferably held by a slight interference fit with the recess or the mounting projection 174. The mounting projections 174 and holders 114 are removably held together by a slight interference fit so manipulation of the top 166 moves and positions all three holders together. The top and bottom portions 166, 168 are then placed together with the holders 114 fitting into the recesses 178 of the container 164. The centerline of the recesses 172, 178 coincide with centerline 115 of the holder 114. The recesses 172, 178 join to form compartments and within each compartment a holder 114 and its associated probe 118 are held. Air can flow through openings 175, 179 to dry the absorbent probe 18, 118 on the holder 114 held within the container 164. The ribs 124 extend sufficiently along the length of the holder 114 so that they position the holder inside the recess 178 and help avoid the probe 118 hitting the sides of the compartment that includes recess 178. The snap lock portions 170a, 170b on top 166 and bottom 168 engage to releasably hold the top and bottom portions of container 164 together.

Advantageously, under aseptic conditions the holders 114 (with their probes 118) are initially placed by machines (e.g., robotic manipulators) onto the mounting protrusions 174. A slight interference fit is used to securely but removably fasten the holders 114 to the protrusions 174. The top (with holders 114) and bottom portions 166, 168 are then fit together manually or by machines, such as robot manipulators. Thereafter, a series of ejectors, one for each recess 172, and having one or more fingers aligned with the openings 175, are passed through the openings 175 to push the holder 114 off of the mounting projection 174 so the flange 125 abuts the top of the wall forming recess 178 in the bottom portion 168. This is done under aseptic conditions. If any chemicals are to be added to the absorbent probe 114, such as a surfactant, reference standard, anticoagulant, stabilizer (e.g., inhibitor enzymes), modifier (e.g., Betaglucuronidase), etc., it is preferably added before the holder 114 is positioned on the mounting protrusion, but could be added before the top and bottom portions 166, 168 are fit together. Such chemical addition is preferably done under aseptic conditions. The assembled container 164 with holders 114 in each compartment may then be placed in a sterile bag for shipment to the user. The bag is optional.

In use, a user unfastens the releasable lock 170 and removes the top portion 166 of container 164. Since the manipulating end of the holder 114 was pushed out of interfering engagement with the mounting projection 174 the lid or top 166 may be readily removed without removing the holders 114. The user may remove each holder 114 separately to directly acquire a sample using the probe 18, 118. Since the manipulating end of the holder 114 was pushed out of interfering engagement with the mounting projection 174 the holders rest in the bottom portion 168 of the container by gravity and may be easily removed by the user with one hand. After sampling, the holder 114 and probe may be placed in a drying rack, or advantageously placed back in the recess 178 of the container 164. A portion of the holder 114 abuts the container 164 to position the absorbent probe 18, 118 adjacent to, but not in contact with, the bottom 179 and its air openings 180. The abutting portion or positioning limit may be flange 125 (FIG. 18a), or it may be a notch 123 in one of the ribs 124 (FIG. 18a), or it may be another surface on the outer surface of the holder 114. Three holders 114 are preferred in order provide one sample for analysis, one as a backup if the initial test goes wrong, and one may be used for future verification or retesting. But different combinations of holders may be provided in kits or containers of various quantities.

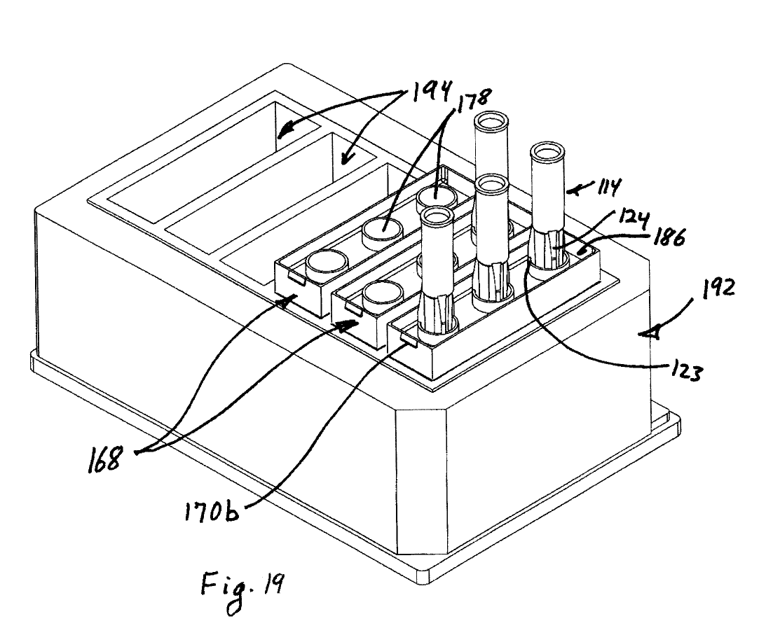

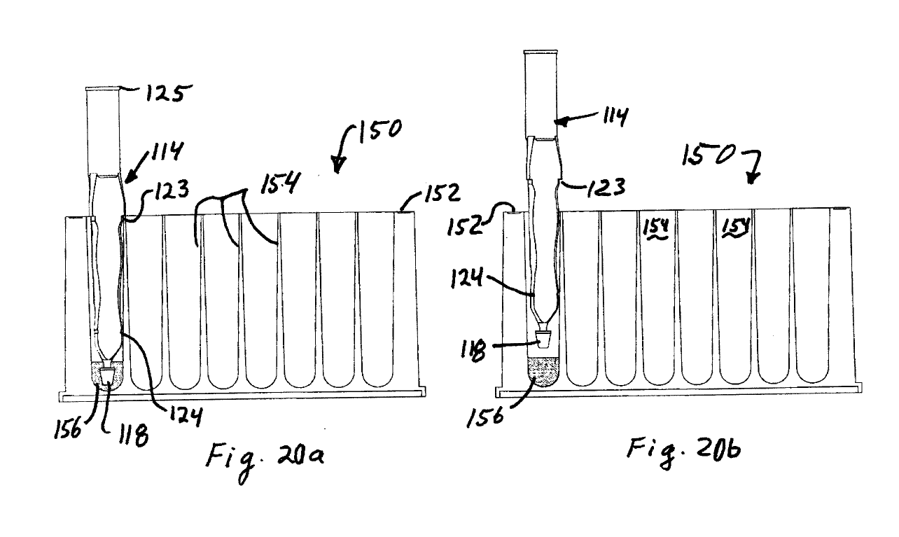

Referring to FIGS. 19-20, for large scale sampling operations it may be desirable to have a plurality of containers 164 and their holders 114 available. A base 192 may be provided with a plurality of recesses 194 configured to receive the bottom portion 168 of the container 164. This base 192 may be used during sampling, or after sampling at the processing laboratory, or for drying. If used for drying, the base 192 may be heated, as for example, by heating coils in the bottom of the base or sidewalls of recess 194 adjacent absorbent probes 118. The base 192 and containers 164 are advantageously configured to locate the holders at predetermined locations suitable for robotic manipulation. Spacing the centerlines of the holders 114 at about 18 mm apart is believed suitable for this purpose.

A series of common numbers, letters etc. are applied to the container 164 and each holder 114 within the container to identify them as corresponding to the same subject or patient and make it easier to coordinate results if individual holders 114 become separated during sampling or analysis. As seen best in FIGS. 15a, 15b, 16c and 16f, the top 166 of the container 164 has an access port 182 on at least one side of the top 166, with one access port aligned with each recess 172. Rectangular shaped access ports 182 are shown, but the shape can vary. The access ports 182 are sized and configured to allow visible indicia to be applied to the holders through the ports 182.

When the holders 114 are held on the mounting probes 174 and the top and bottom portions 166, 168 of the container 164 are assembled to enclose the holders and probes 18, 118, the access port 182 allows access to the outside of the holder through the port. Thus, when the holders 114 and probes 18, 118 are packaged for shipment in container 164, identifying indicia 181 (FIG. 13a, 15a, 15b) can be affixed to each holder 114 and to the container 164 or top 166. The identification indicia are advantageously a serial number associating each holder 114 in the container 164 with the other holders in the container 164 and with that container. While printed indicia printed on the holder 114 is preferred for indicia 181, adhesive labels are also believed suitable as are other mechanisms for providing visible indicia to the holders. Advantageously, the ribs 124 on the holder 114 do not extend to the end of the holder adjacent flange 125 which is opposite the probe 18, 118 and thus the end of the holder has a generally smooth and preferably cylindrical outer surface which can readily accommodate printed indicia or labels 181 as applied through access ports 182. The access ports 182 thus extend along a sufficient length of the top 166 to allow the visible indicia to be applied through each port to the holder 114 aligned with or corresponding to each access port. The access port 182 allows air passage into the recess 172, 173 of the container 164 and helps dry the absorbent probes 18, 118 when the container is closed. The bottom part 168 of the container preferably does not have any openings, but could have some if it believed desirable, for example, for drying of probes 118.

As best seen in FIGS. 16c and 16f, the top 166 has a recessed portion extending around its periphery to form an offset male projection 184. The bottom 168 has a correspondingly configured recess 186 (FIGS. 17b, 17f) on its inner periphery shaped to mate with the projection 184 in the top 166 to better hold those parts together. The male and female mating projection 184 and recess 186 could be on the opposite parts. On an outward facing portion of the inset, male projection 184 there are preferably visible indicia 188 which identify the recesses 172 and holders 114 therein. The indicia 188 preferably comprise numbers such as numerals 1, 2 and 3, or letters or other simple designations associated with a different one of the sequential recesses 172 and the corresponding mounting projections 174. The indicia 188 may be molded with the formation of the top 166, or it may be printed, or otherwise applied. The indicia 188 help the user associate a specific holder 114 with its mounting projection 174 and recess 172. The indicia 188 on the top 166 is preferably associated with the visible indicia 181 on the holders 114 so a user can more easily remove a holder 114 from its associated recess 172 and mounting projection 174, use its associated probe 18, 118 and then return the holder to the same recess 172 and mounting projection 174. Advantageously, a portion or all of indicia 181 is contained in indicia 188, or vice versa.

By pushing the holder down into the recess 172 and along the length of the mounting projection 174 the user can wedge the holder in place on the top 166, preferably by an interference fit with the ribs 176 on mounting projection 174, but alternatively by an interference fit with the walls forming recess 172 in the top 166. Wedging the holder 114 in place not only helps releasably fasten the holder to the top 166, bit it helps align the holder with projections 174 and that makes it easier to insert the holders into the bottom 168 of the container 164.

Referring to FIGS. 12 and 20a-20b, when container 134, 164 is received at a laboratory or processing location, the holder 114 and associated absorbent probe 118 are removed from the container and placed in a well plate 150 by manually or robotically grabbing the end of the holder opposite the probe 118 or by inserting pipette handling equipment into the open end of the holder, or by robotic handling equipment. The well plate 150 conforms to SBS Microwell plate specifications and has a top wall 152 with plurality of tubular recesses 154 opening onto that top wall. The recesses 154 are typically cylindrical in shape, often with tapered, closed ends. Advantageously the flange 125 or notch 123 on holder 114 is sized so that it abuts the top wall 152 to position the absorbent probe 118 adjacent the bottom of the recesses 154, with flange 123 and ribs 124 being sized relative to the diameter of recess 154 to limit lateral motion of the holder 114 in the recess. Thus, the holder 114 is inserted into a recess 154 of the well plate 150. The length of the holder 114 and the location of the flange or notch 123 may be selected to position the absorbent probe 118 at a desired position within the recess 154 of well plate 150. The flange 123 may be omitted in which event the ribs 124 cooperate with the walls forming recesses 154 to keep the dried absorbent probe 118 centered in the recess and away from the recess walls during processing. Instead of a well plate 150, the holder 114 and probe 118 could be placed in a single tubular container.

Once the holder 114 and absorbent probe 118 are positioned in the recess 154 of well plate 150, suitable extraction fluids 156 are added to the recess 154. The fluids 156 may be in the recess 154 before the holder and probe are placed in the recess. Typically, the well plate will be vortexed, sonicated or otherwise agitated to intermix the fluids 156 and (dried) blood on the probe 118 in order to help extract the blood from the probe. If a flange 123 (FIG. 8) is used on the holder 114 the flange can act as a cap and/or splash guard during extraction vortexing, sonnication or agitation. Since vortexing may cause the solvent to climb the walls of recess 154 the flange 122 may optionally be provided above the maximum height of the vortexed fluid in order to avoid the back side of the flange from collecting the intermixed fluids and impeding complete recovery of the sample. Alternatively, the flange 123 may be placed close enough to the wall of the recess 154 to disrupt vortexed fluid from climbing the wall.

After the dried blood or other sample fluid on probe 118 is extracted by extraction fluid 156, the fluid is removed by various means through the top or bottom of recess 154. The holder 114 and probe 118 are typically removed from the well plate 150 and recess 154 to allow access to the fluid therein for easier removal of the fluid, or in some instances for further processing of the fluid within the recesses 154. The holder and probe may then be discarded, or retained according to specific needs. The fluid 156 with the sample extracted from absorbent probe 118 is then further processed to further analyze the sample.

This above method and apparatus are especially useful for testing of biological fluids, especially for sampling blood for use in testing for either research or for diagnostic use. The simplified method includes placing the absorbent probe 18, 118 in contact with the fluid to be absorbed and allowing the sample to be absorbed by the probe, preferably by manual manipulation of the probe and the holder connected to the probe. The fluid sampled does not have to be free of or separated from red blood cells (plasma or serum). Indeed, the absorbent probe 18, 118 is preferably used to absorb fluid directly from the sample, and is believed particularly useful for absorbing whole blood from a pricked finger. Thus, the probe 18, 118 advantageously absorbs both the liquid portion of blood (plasma) as well as the red blood cells.

The probe 18, 118 is used to directly contact the source of fluid to be sampled. This differs from prior art devices that used capillaries or narrow filtering passages to contact a fluid source and connect to a fluid retaining matrix or cavity. By directly contacting the fluid source with the sorbent probe 18, 118 the uptake or absorption of fluid is increased and the time to do so is reduced. Thus, advantageously a majority (over 50%) of the surface area of the absorbent probe 18, 118 is exposed and available for both absorbing fluid and allowing access to air and gasses to dry previously absorbed fluid. The material selected for the probe 18, 118 is thus both fluid permeable to increase absorption rates, but also gas permeable to increase drying rates and shorten drying times. Preferably, a substantial majority (over 80% and preferably over 90%) of the surface of probe 18, 118 is available for contact with the source of fluid and available for drying absorbed fluid. By having such a large surface area available for absorption and drying, the ease of manipulating the absorbent probe 118, positioning the probe relative to the fluid 22, and the ease of contacting the fluid with the probe are all greatly increased. The large portion of exposed surface also helps shorten the drying time.

Referring to FIG. 9, the probe has a length L extending along a first, longitudinal axis 115, and sides surrounding that axis with the sides being of various shapes, including curves, planes or combinations thereof and being of various number. Preferably the shape is selected or the probe configured so the absorbed fluid 22 travels about the same distance into the probe regardless of where the fluid contacts the surface of the probe 18, 118. Thus, the exterior surfaces of probe 18, 118 orthogonal to the longitudinal axis L are preferably about the same, say within about 20% of the axis 115.

Referring to FIG. 14c, the holder 114 is preferably tubular adjacent the end of the holder opposite the probe 118. The recess 190 forming the tubular shape may extend entirely through to the holder 114. That construction allows solvent to be poured into the recess 120 and pass from inside the holder through the probe 118 and out the outer surface of the probe in order to remove previously absorbed and dried fluid 22 from the probe. A passageway with a circular cross-section that is constant, or preferably that tapers slightly along the length L of the holder 114 is believed preferable. In the configuration of FIG. 14c, the outer periphery of the probe 118 is placed in the opening at the end of the passageway or recess 190 and preferably press fit into position to block the opening and hold the absorbent probe 118. Alternatively, suitable adhesives may connect the parts, or mechanical fastening means such as small hooks or deformations of the holder 114 that extend inward toward axis 115 and are located around the opening in the probe-end of the holder 114 could be used to create an interference fit between the tubular tip of the holder and the abutting periphery of the probe 118.

As seen best in FIGS. 12, 13b, 14c and 18a, the absorbent probe 18, 118 has an exterior surface that is preferably fully exposed so that except for the connection to the holder 14, 114 the surface of the probe is exposed and available for contacting with the fluid to be sampled. It is believed to make the probe 18, 118 elongated with a connection to holder 4, 114 at one end of the probe. Advantageously, the connection of the probe 18, 118 to the holder 4 is such that less than about 25% and preferably less than about 15% and more preferably less than about 10% of the surface area is blocked by the connection and not exposed for directly contacting the fluid to be absorbed. Likewise, the surface of the absorbent probe 118 is not sheathed or shielded by any material that would prevent absorption of fluid 22 during use, or that would impede access of air or other gas to dry the fluid absorbed by absorbent probe 114.

The pyrolyzed material used for the absorbent probe 18, 118 should be hydrophilic. The material may initially be hydrophobic or hydrophilic and treated to make it hydrophilic. Hydrophobic matrices may be rendered hydrophilic by a variety of known methods. Among those methods available are plasma treatment or surfactant treatment and those methods are believed suitable for use with the carbonized matrix. Preferably, plasma treatment is believed suitable to render the pyrolyzed porous carbon hydrophilic or to improve its hydrophilic properties.

Surfactant treatment involves dipping a hydrophobic matrix in a surfactant and letting it dry. This surfactant treatment assists in wetting the surface and interior of the matrix and results in the promotion of aqueous liquid flow through the matrix. It is contemplated that a wide variety of commercially available surfactant materials would be appropriate for use with the present invention. The surfactant treatment has the disadvantage of potentially adversely affecting later processing of the sorbent 18, 118 and fluids retained therein, depending on the particular analyte, solvents and analysis involved. The surfactant is thus preferably chemically stable relative to the fluid being sampled. If that fluid is blood, the treatment of such hydrophilic material to make it chemically stable (e.g., by pre-adsorbing a surfactant such as Triton X) can lead to interference in the analysis of the sampled or absorbed fluid, so the specific surfactant used may limit the use of the probes 18, 118. Note that surfactants are preferably adsorbed onto surfaces of the probe rather than absorbed into the probe.

In general, surfactants should be selected which are compatible with the reactants or reagents placed within the matrix so as not to interfere with the preferred activity. Additionally, it should be noted that no surfactant should be present in such concentrations as to cause hemolysis of the red blood cells. In addition, care must be exercised to avoid hemodilution of the plasma sample. Hemodilution is the extraction into the plasma of the internal fluid of the red blood cell due to hypertonic conditions.

The material used for probe 18, 118 advantageously has a predetermined porosity and void space in the open cell structure of the pyrolyzed porous carbon material. The porous material will retain fluid in its interstices in proportion to the volume of the porous matrix. A pore size of from about 10 microns to about 80 microns is believed especially useful for biological fluids like blood. Such a pore size allows individual red blood cells to pass readily into the probe material. If the pore sizes are too small, then the time to absorb a predetermined sample volume will increase. The material and its porosity and pore size must be reproducible in order to provide a reproducible fluid uptake capacity of the probe 18, 118.

The treatment of the material used for the probe 18, 118 can also impart an ionic character to the probe material (or probe) which could be advantageous in selective adsorption and enrichment of analyte molecules. This added ionic character could be positive or negative charge, or specific chemical moieties such as phenyl, hydroxyl, or other groups that are believed to improve selectivity or retention for the analyte molecule(s) used in blood analysis and testing.

The probe 18, 118 could also be manufactured to entrap chromatographic particles with various desired chemical properties in order to allow for selective retention or enrichment of the analyte(s). The chromatographic particles would be added to the mold when manufacturing the probe in a desired concentration and be entrapped within the porous network of the probe material, in this case—pyrolized porous plastic.

The volume of the probe 18, 118 is advantageously kept small, just large enough to absorb about 30 microliters of a fluid sample 20, advantageously just large enough to absorb about 20 microliters of fluid sample 20, and preferably large enough to absorb about 10 microliters. Devices 10 sized accordingly are believed preferable, with a multi-volume device 10 having a probe 18, 118 sized to absorb about 5-20 microliters being believed desirable for multipurpose use. By keeping the probe 18, 118 and absorbed sample 20 small, several advantages can be achieved.

First, the absorption time is short since the volume to be absorbed is small and since the material of probe 18, 118 is selected to absorb fluid rapidly. The absorption is further increased when the majority (over 50%) or substantial majority (over 80%) of the entire surface of the probe 18, 118 is exposed for potential contact with the fluid sample 20.

Second, the small volume of the absorbed fluid sample 20 allows the sample to dry faster. Since biological samples degrade analytes, and since dehydrating the sample and analyte retards degradation, fast drying helps slow down the sample degradation. For example, if the desired analyte is a specific drug, enzymes in blood may degrade one or more drugs or analytes sought to be detected by testing. Drying the blood quickly helps slow down the degradation. Drying a small sample on probe 18, 118 are faster than drying a large sample. To reduce drying time, the material used for the probe 18, 118 is preferably selected to be air permeable or gas permeable so that air can enter the probe 18, 118 and dry it faster.

Third, dried biological samples are generally not classified as bio-hazardous materials and may be shipped through the mail, etc. That makes it easier for shipping and handling, and costs less than shipping fluid samples. A shortened drying time also allows more samples to be taken, dried, packaged and shipped per unit time, thus increasing efficiency and reducing costs. Fifth, small samples may be extracted faster from the probe 18, 118. Using devices 10 to allow and facilitate robotic handling also reduces time and costs of the analysis. Using probes 18, 118 configured for easy placement in analytical tubes, or having internal passageways for solvents to pass through the probes to extract the dried samples further reduces the extraction time. Sixth, the small probes 18, 118 leave less material for disposal. This is especially useful if the probes 18, 118 from which samples are extracted are still considered bio-hazardous materials. Seventh, the probes 18, 118 from which solvents have extracted the sample, may be dried more quickly, thereby making them more easily to handle, discard or destroy than wet absorbent materials.

The shape of the absorbent probe 18, 118 will vary and is preferably optimized to improve wicking speed. However the tip diameter of the probe need not be any larger than the diameter of a 30 ul spot of blood. A probe 18, 118 with a circular tip diameter of about 0.1 inches (0.25 mm) is believed suitable. A truncated, conical probe 18, 118 having a further length of about 0.16 inches (4 mm) and a base diameter of about 0.14 inches (about 3.5 mm) is believed suitable. The surface area of the probe in contact with the blood is preferably maximized and thus the sides and tip of the probe 18, 118 advantageously present an exterior surface area of about 59 mm (0.1 in²). The area is preferably sufficient to contact the entire area occupied by a 30 microliter sample of blood on the surface on which the wound 23 is located producing the blood.

Additionally, the use of anticoagulants during the collection of blood may be useful in maintaining the homogeneity of the blood as well as preventing unwanted degradation. The addition of dried anticoagulants to the probe 18, 118 may help prevent these unwanted effects, An anticoagulant may be applied dry to the probe 18, 118 but is preferably applied wet or in liquid form and allowed to dry before use. Any anticoagulant applied to the probe 18, 118 is preferably selected for use with any anticoagulants in the matrix of any reference standards that are used, and is selected to be compatible with any fluids used in extracting the analytes from the dried blood or sample on the probe 18, 118. The most common anticoagulants fall into two categories polyanions (e.g. Heparin) or metal chelators (e.g. EDTA, citrate). Suitable anticoagulants are believed to include acid citrate dextrose, citrate phosphate dextrose, citrate phosphate dextrose adenine, sodium citrate, K2 EDTA, K3 EDTA, sodium EDTA, lithium heparin, sodium heparin, potassium and oxalate. Any dried anticoagulant applied to probe 18, 118 should be suitably matched with the extraction fluids and downstream analysis so as not to adversely affect the accuracy of the analysis.

The use of internal and external standards during analysis is common practice and a reference standard (wet or dried) may be applied to the absorbent probe 18, 118 during manufacture or sampling of the fluid, or it may be added to the reconstituting fluid when the dried blood or other fluid is extracted from the absorbent probe 18, 118. Many non-volatile materials which do not affect the analysis of the blood or fluid may be used as reference standards. Radiolabels, fluorescent labels, deuterated labels may be used. For example, during extraction of the probe an analyst may add a standard for the analyte of interest to the extraction solvent. For ease of use, standards can also be dried onto the surface of the probe prior to use or after a sample has been collected and dried onto the probe, thereby eliminating the need to add internal standards to the extraction solvent. Additionally, a set of probes 18, 118 may be made with reference standards of dried blood that are to be processed along with the collected standards in order to check the extraction and/or analytical processes or to provide a reference for the extraction and/or analysis.

Additional treatments of the absorbent probe 18, 118 may be useful for the analysis of specific types of biological molecules such as proteins and nucleic acids. For each analysis, improving the stability of the molecules to be analyzed or preparing the molecule for analysis during drying and storage can improve later analysis. As an example of stability improvements, in the case of protein and peptide analysis it is useful to deactivate other proteins such as proteases which chemically degrade both proteins and peptides. Mixtures of protease inhibitors (and inhibiting molecules such as Urea and Salts) can be dried onto the surface or interior of probe 18, 118 so that proteins and peptides are stabilized during drying and storage. Likewise, in the case of nucleic acid analysis it can be valuable dry additives such as salts, chelators, enzymes which degrade nucleases (such as proteinase K) to prevent the activity of molecules that degrade nucleic acids. In the case of drugs and small molecules they are commonly metabolized into Glucuronides during conjugation for excretion. In the example of urine analysis it can be useful for later analysis to incorporate Beta-glucuronidases enzymes into the probe 18, 118, which will convert the drug for analysis back into its original form.

Not only is the absorbent probe 18, 118 useful for fast absorption of fluids such as blood, but the material of the probe also decreases the drying time. Since enzymes in bodily fluids such as blood deteriorate the samples and drying renders the enzymes inactive. Further, the probe may be pre-treated with a material to retard enzyme action on the absorbed blood. Applying Urea to the probe and drying it is believed useful for enzyme inhibition. Applying a weak acid is also believed suitable if the acid is selected so it does not degrade the absorbed sample. Various protease inhibitors and inhibitor cocktails are available and could be applied to and dried on the probe 18, 118. For example, one protease inhibitor provided by Sigma Aldrich uses a combination of AEBSF (2 mM), Aprotinin (0.3 μM), Bestatin (130 μM), EDTA (1 mM), E-64 (14 μM) and Leupeptin (1 μM).

One purpose for these treatments is to prepare the sample for analysis, or to eliminate steps prior to analysis. Another common method of sample preparation is solid-phase extraction. The structure of the probe and the methods used in forming the probe 18, 118 allow for incorporation of sorbent particles (both silica and polymeric) that can capture analytes of interests during the drying step and then release them only under specific extraction conditions. Due to the specific nature of the extraction conditions, the probe can be washed with a variety of solvents that will remove interfering components from the biological fluid on the probe 18, 118. Then when the analyte of interest is extracted from the probe the sample that is extracted will be free of interfering biological matrix components.

As an example, microspheres in the 20-50 micron size range can be incorporated into the probe 18, 118 during formulation of the probe or by treatment after formation. Other sizes of particles may be used depending on the application, with microspheres as of about 120 microns in diameter being believed suitable. These microspheres could contain a high density of hydrophobic ligands on their surface, and will interact strongly with hydrophobic analytes such as Vitamin-D and its metabolites. When blood is collected onto such a treated probe 18, 118, the free analyte will partition onto the hydrophobic surface. During extraction, the analyte can only be extracted from the probe with non-polar solvents. So, the probe can be washed with aqueous solvents or mixtures of aqueous and organic solvents without removing the hydrophobic analyte. When washing is complete the hydrophobic analyte can be eluted with a strong organic extraction solvent.

The above description maintains the probe 18, 118 on the holder 114 during use. It is believed possible to remove the absorbent probe 18, 118 from the holder for extraction of the sample and analysis, but that is not believed as efficient from a time viewpoint. The holder 114 and probe are a single unit handled by the user, and have no individual protective sheath enclosing all or a substantial portion of the holder, and do not have the holder 114 reciprocating within an enclosing protective cover during use.

The porous probe 18, 118 of carbonized material provides a ready method of collecting a sample for analysis, which method includes contacting the porous probe with a liquid sample to be collected and allowing the liquid sample to be absorbed into the porous probe. The method may also include allowing the sample to dry on the porous probe. The holder 14 may comprise a retractable holder like a spring-loaded, ball point pen using the probe 18, 118 in place of the cylindrical ink cartridge, in which case the method would include retracting the probe into the holder body or placing a protective cap (preferably of plastic) over the probe and then retracting the probe into the holder body. The method further includes releasing the porous probe into a container and extracting an analyte from the probe with a solvent or a solution. The method may also include detecting the analyte in the extracted solution. The step of detecting the analyte in the extracted solution may be performed at least in part by using a mass spectrometer.

In further variations, the method may include wetting the porous probe containing the dried sample with a solution, then applying an electrical potential to the wet porous probe and then detecting ionized analytes released from the wet porous probe using a mass spectrometer. Similarly, the method may include the steps of wetting the porous probe containing the dried sample with a solution, then applying the wet porous probe to a surface to transfer sample from the wet porous probe to the surface and the introducing the surface into a mass spectrometer which may be used to detect ionized analytes released from the probe. The methods disclosed herein may also include treating the probe to increase its hydrophilic properties.

A lancet for puncturing the skin may be provided with the kits described herein, and the method of use may involve punching the skin to enable a drop or pool of blood to be accessed by the absorbent probe. Thus, the method may include piercing the skin of an animal with the lancet to release blood, contacting the blood with the porous probe 18, 118, allowing the blood to be absorbed into the porous probe and then allowing the blood to dry on the porous probe. This method may include the above described steps, including the step of capping the porous probe with a custom fitted cap or by placing the probe in the container. The above devices and methods may include configuring the probe 18, 118 to absorb and using the probe to absorb from about 1 μl to about 250 μl, and more preferably configure the probe to absorb and using the probe to absorb about 10 μl to about 100 μl, and still more preferably to absorb 1 μl to about 25 μl. The probe 18, 118 is advantageously short, preferably about 5 mm in length to absorb up to about 10 μl of fluid.

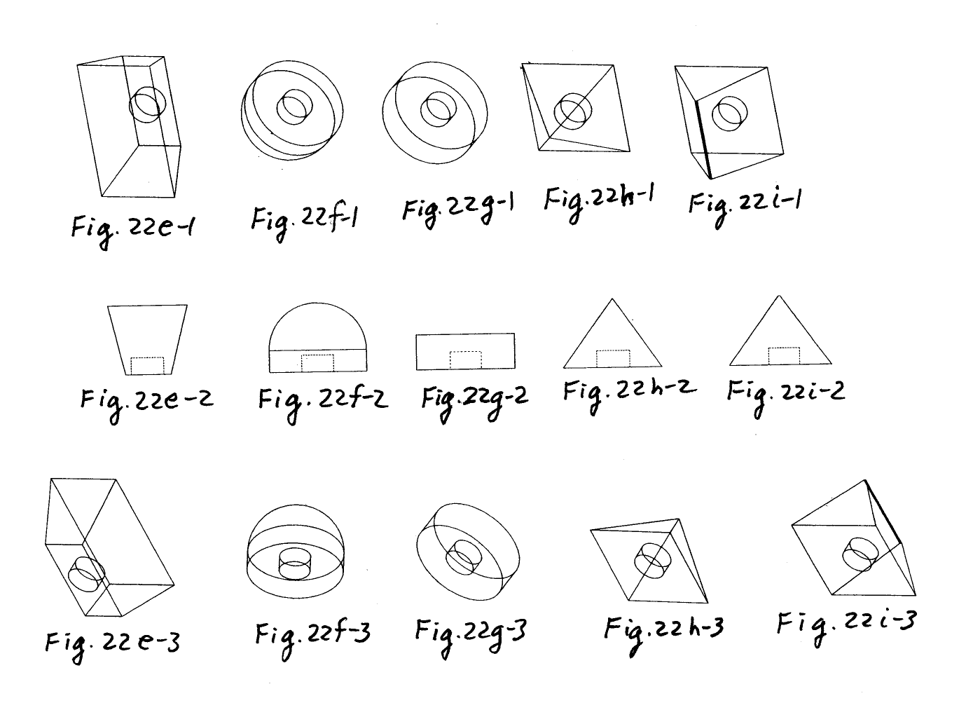

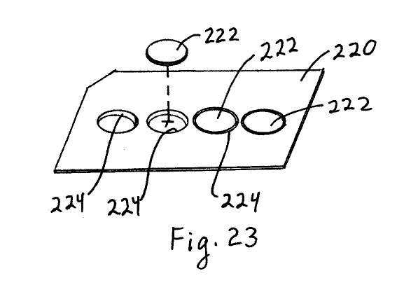

Referring to FIG. 23, a to ‘blood spot card’ 220 is provided patterned after conventional cards, such as Whatman's FDA Eulte, using 15 ul aliquots spots at four locations represented by discs 222. Each of the spots or discs 222 preferably comprise a disc of porous material used to form the porous probe 18, 118, with each disc 222 being cut to the desired diameter and thickness and then press-fit into the card 220, with the card 220 made of paper, plastic or other suitable material. Each disc 222 preferably has a flat upper and lower surface and a periphery that is slightly larger than the holes 224 formed in the card 220 to receive the disc. The holes 224 in the card may be punched, cut or formed by other means known to one skilled in the art. It is believed possible, but less desirable, to form the entire card out of the material used to form porous probe 18, 118 and then mark the absorbent area with a circle as is the current practice. In either case, the fluid sample 22 is placed in contact with the material in the disc 222 and absorbed. The sample 22 is then dried on the card 220, with the card packaged, shipped and received at the testing location. The disc 222 is removed from the card 220 by manually pressing out of the disc 220, or by punching the disc from the card or by automated punching or pressing or other removal techniques. The removed disc 222 and the dried sample 22 is reconstituted and tested as desired. In short, the disc 222 is handled as is the probe 18, 118, with the card 220 acting as the holder for the probe.

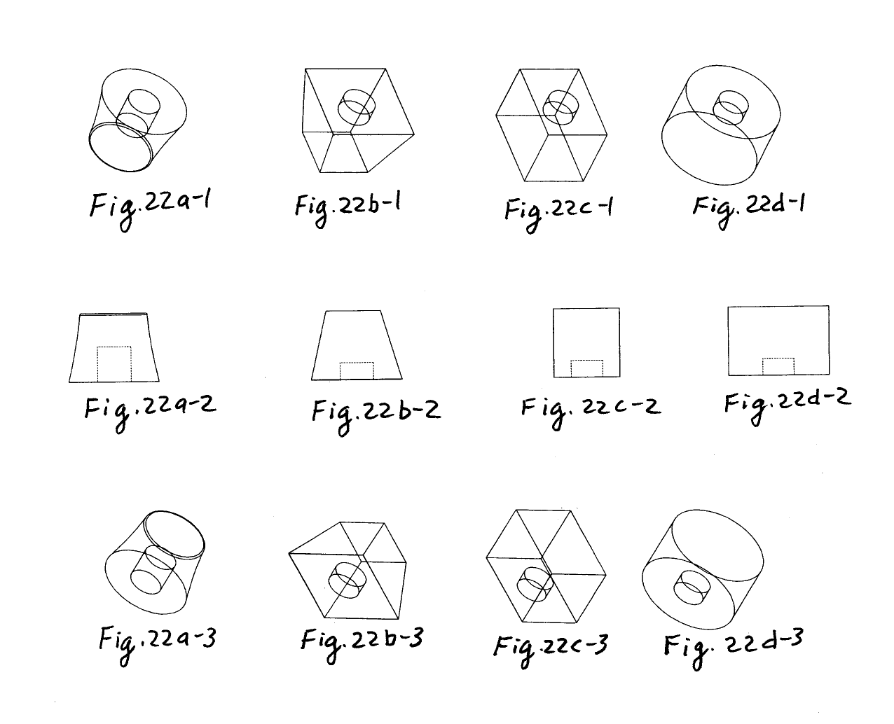

Referring to FIGS. 22a-22i, the porous probe 18, 118 may take various forms with the volume selected to absorb a predetermined fluid volume of liquid sample 22 and with the external shape configured to expedite the absorption of that material while minimizing the volume of liquid sample that is not absorbed and “hanging on” to the external surface of the probe. A shape that has few and preferably no concave exterior surfaces is preferred. Since the probe is made of hollow particles the lack of concave exterior surfaces refers to the gross, exterior shape rather than that of the material forming the overall shape. The depicted shapes include cones, truncated cones, quadrilateral shapes, cylindrical shapes, spherical shapes, domed shapes, disk shapes, pyramidal shapes, and other surfaces with no concave surfaces. Other shapes may be used. The various shapes shown in FIGS. 22a-228, and the tip shapes shown in other figures of this application, comprise absorbent means configured for absorbing a predetermined volume of fluid.

Referring to FIGS. 24-25, a cross-section of a distal end of a holder 114 is shown with holder tip 230 onto which a specific embodiment of the absorbent probe 18, 118 may be connected. In the depicted embodiment the holder tip 230 is preferably, but optionally cylindrical with a rounded end that fits inside a mating recess or cavity in the absorbent probe 18, 118. The holder tip 230 can also have a cylindrical shape with a flat end perpendicular to the axis of the cylindrical tip. The depicted absorbent probe 18 has a bullet shape preferably having a rounded or curved lower end 232 joining a generally cylindrical sidewall 234. But as shown in FIGS. 24a-24c, the distal end 236 may alternatively have a molding artifact typically tanking the form of a short, circular disk with a flat top that is centered on the longitudinal axis with the disk joining outwardly or convexly curved sides 232 that in turn join the generally cylindrical sidewall 234. A distal end 236 that is curved is preferred. The flat end 236 is a forming artifact that results from the expansion of the molded material when the molding pressure is released with the flat disc corresponding to the location of a molding runner or slide. A molding artifact several thousandths of an inch high and a several hundredths of an inch in diameter can result on absorbent tips having a diameters from about 0.14 to 0.24 inches. The juncture of the cylindrical sidewall of the artifact 236 is believed undesirable but tolerable and it is believe any adverse effects of the juncture may be significantly reduced or removed by machining or grinding operations or altering the molding techniques. As used herein, a substantially curved end 236 encompasses this molding artifact.

A recess 238 is formed in the upper end of the absorbent probe. The recess 238 and holder tip 230 are configured to mate with each other and preferably have complementary shapes, with the recess 246 being slightly smaller than the holder tip 230 to create a slight interference fit or press fit to secure the probe to the holder tip.

To accommodate forming tolerances for molded probes, the recess 246 advantageously has a taper of about 0.5° from the longitudinal axis along the recess, on each side of the recess, for an included angle of about 1°. The outer cylindrical wall 234 has an inclination of about 2° from the vertical axis for mold release, so the probe is slightly larger in diameter at the upper end near the opening to recess 238 than it is nearer the curved end 232. The recess may have a flat interior end or a rounded, preferably hemispherical interior end. The edge of the recess 238 may have a chamfer 240, especially on thinner walled absorbent probes.

A maximum outer diameter of the absorbent probe is believed to be about 0.23 inches (about 6 mm) for extraction in a commonly used 96 well plate extraction with wells about 8.5 mm in diameter and spaced about 9 mm apart. But the exact dimensions will vary. The relative dimensions for illustrative absorbent probes are provided in Table 1 below, with dimensions in inches unless noted otherwise.

TABLE 1

| Absorbed | | | | |
|---|---|---|---|---|
| Volume | 100 μl | 150 μl | 200 μl | 300 μl |
| Outer Dia. | .141 | .167 | .180 | .200 |
| Length | .157 | .164 | .184 | .220 |
| Recess Depth | .112 | .121 | .134 | .16 |
| Wall Thickness | .040 | .048 | .05 | .063 |

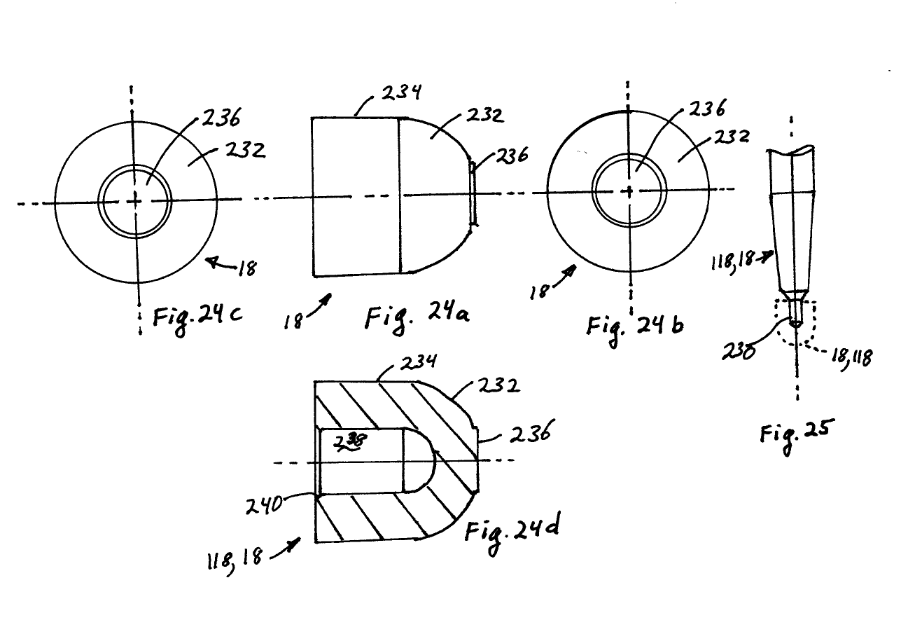

A further embodiment of the absorbent probe 18, 118 is shown in FIGS. 26a-26d, where an inclined, conical surface 242 joins the cylindrical wall 234 to the curved end 232 so that the conical surface 242 has a larger diameter at the juncture with the cylindrical wall 234 than at the juncture with the curved end 232. The conical surface 242 is inclined at an angle of about 26° relative to the longitudinal axis of the absorbent probe and preferably has rounded junctures with the sidewall to form a curved end 232. The molding tolerances, recess 238, chamfer 240 and molding artifact 236 are as previously described and the dimensions are as generally stated in Table I, except for a slight variation in thickness arising from the tapered or conical surface 242.

A frusto-conical absorbent probe with a flat or rounded end is shown in FIGS. 26a-26d. The configuration is similar to the probe of FIGS. 24a-24c and the description of the common parts are not repeated. The curved side 232 (FIG. 24a) is formed by at least one straight side segments, preferably one straight side segment 242a and one curved segment 242b with rounded junctures on each end of the segments to approximate a hemispherical curved surface with straight sided, conical surfaces curving about the longitudinal axis 115. The holder tip 230 is advantageously configured to provide a uniform or a substantially uniform wall thickness of the absorbent probe, and thus may have also have a frusto-conical shape with a rounded end.

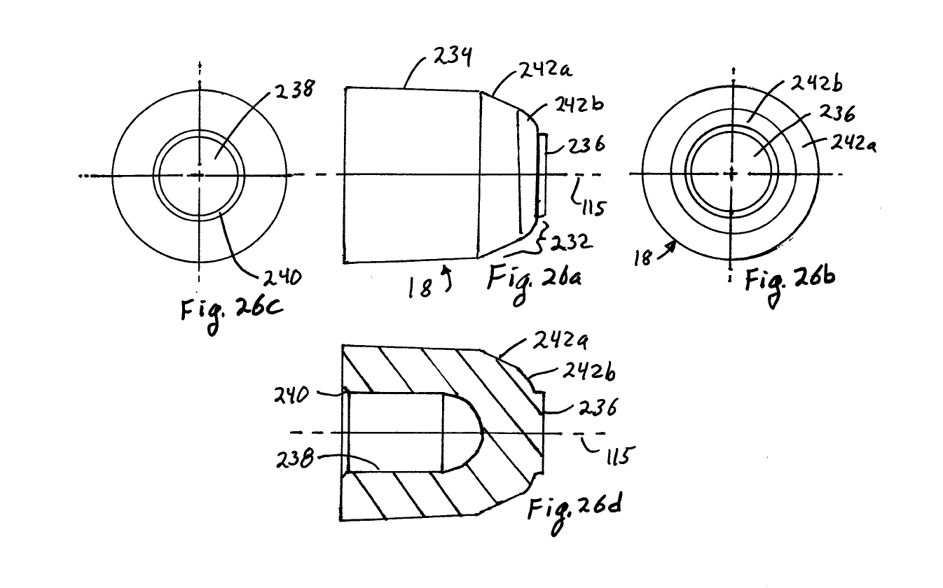

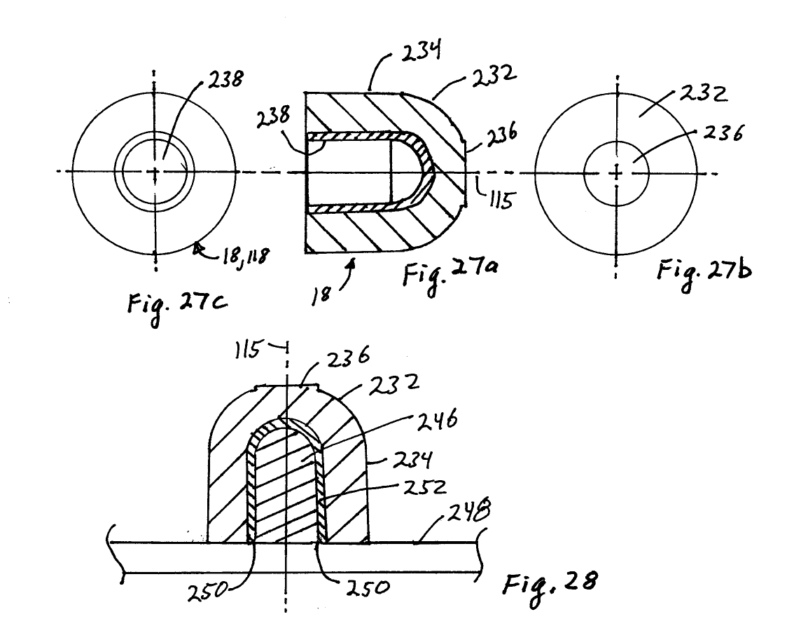

Referring to FIGS. 27a-27c and 28, the thickness of the wall forming probe 18, 118 is preferably thin, from about 0.01 to 0.065 inches. As the wall becomes thinner, especially below 0.02 inches, the wall may be sufficiently thin or flexible that it is difficult to form the probe on a very small diameter tip holder 230 over which the probe is formed or placed for use, and it is difficult to form the thin wall even on a larger core pin when the wall thickness becomes too thin. An over-molding forming technique may be used to form an over-molded probe 18, 118 having a non-porous inner portion providing handling strength and stability, and a thin, porous outer portion for absorbing fluids. This over-molded porous probe is believed suitable for the sintered plastic probes but not preferred for the porous carbon probes.

A core pin or molding post 246 (FIG. 28) is fastened to a support plate 248, with a large number of such core pins 246 typically being used and extending perpendicular from the plate. As needed, bosses 250 are provided around the base of the support post in order to form the chamfer 240. The core pin 246 and boss 250 have the configuration of the desired recess and chamfer 238, 240 in the finished absorbent probe 18, 118. An inner layer 252 is over-molded onto the core pin 246 with the inner layer with the inner layer dimensions being selected to provide a desired wall thickness on the final probe 18, 118. The absorbent probe material is then formed onto the over-molded inner layer 252 by sintering the plastic thereto. It is believed possible to form the inner layer 252 and the porous layer 18 at the same time by using two different materials and/or two different sized particles, one to form the denser, non-porous inner layer 252 and another to form the pyrolized porous portion of the probe 18, 118, with the materials and sizes being selected so that the same temperature and pressure and time will result in a non-porous inner layer and a porous outer layer. The inner layer 252 is preferably nonporous while the material of the outer layer is the porous absorbing materials made of plastic as discussed herein. Inner layers 252 made of the same plastics as described for the plastic porous material are believed suitable for the inner layer 252.

The absorbent probe 18, 118 is preferably configured to provide a substantially uniform wall thickness where the wall thickness is the shortest distance between the recess 238 and the outer surface of the absorbent probe, with the over-molded inner layer 252 being used to provide a sufficient thick wall for manufacturing while allowing a thin absorbent wall thickness. A slight increase in thickness of about 10-15% at the lower or distal end of the probe is believed acceptable and is encompassed by the “substantially uniform thickness” as used herein. The probes are advantageously configured to avoid concave exterior surfaces in order to reduce the tendency of a fluid sample 20 to “hang on” through surface tension or other fluid properties, to the outer surface of the probe. The fluid sample “absorbed” into the body of the probe is desirable until the probe is saturated or full of the fluid sample. The fluid that is “adsorbed” onto or hangs onto the outer surface of the probe when the probe is saturated is undesirable as it may cause problems because it may remain clinging or hanging on the outer surface of the probe as the sample is dried. When the sample dries the extra fluid also dries and cakes onto the outer surface of the probe. That adsorbed fluid that dries increases the volume of fluid over the predetermined volume of fluid the probe was configured to hold when saturated. Small variations in adsorbed fluid have larger effects when the volumes of absorbed fluids are small.

The absorbent probe is preferably configured to have walls that are uniform in thickness in order increase extraction rates and efficiency. The absorbed sample fluids dry by evaporating at the air-fluid interface which is on the outside or exterior of the absorbent probe 18, 118. Thus, the adsorbed, hanging-on material dries faster than the fluid sample on the interior of the probe. The fluid sample is drawn toward the air-fluid interface as fluid on the exterior of the probe evaporates and dries. The result is a higher concentration of dried sample adjacent the outer or exterior surface of the probe, and a reduced concentration of dried sample on the innermost portions of the probe. When the sample fluid is blood or other bodily fluids, the drying process can produce a harder cake material that is more difficult to dissolve in the extracting fluid. When the saturated probe is thicker at one portion than another it absorbs more fluid and thus more of that fluid is drawn toward the adjacent surface as the surface dries, and the result is to form a thicker cake adjacent the surface of the thicker portions of the probe. A more uniform wall thickness is thus desirable because it provides a more uniform caking of the dried sample adjacent the exterior surface of the absorbent probe. A thinner wall thickness on the probe is thus desirable to shorten the drying time of the sample, to improve the dispersion of the dried sample in the probe, to shorten the dissolving and extraction time for the dried sample, and to avoid large volumes of caking and incomplete dissolving of that dried, caked sample during extraction. A thinner wall thickness absorbs fluid at a slower rate so it is counter-intuitive to use a thinner wall probe in applications where short absorption times are desirable, as in collecting blood or bodily fluids from live animals, especially humans.

The distal end of the holder tip 230 is preferably curved because as the wall thickness of the probe becomes thinner, the absorbed fluid does not move as quickly around sharp corners or sharp edges as it does around curved corners or curved edges. For absorbent probes having a wall thickness of about 0.040 inches or smaller the holder tip 230 is preferably rounded and forms a hemispherical end. Any empty space between the wall of the recess 238 and the holder tip 230 may be filled with sample fluid and alter the intended volume held in the absorbent probe. Thus, the holder tip 230 advantageously conforms to the shape of the recess 238 and fills that recess.

In use, the absorbent tip 202 is press fit onto the holder's tip 230. The tip is made of various absorbent materials discussed herein. As needed, the tip 230 may have ridges or barbs (not shown) on its outer surface to resist easy removal of the absorbent tip 202 from the tip 230, but such surface disruptions are preferably kept small when the walls of the probe are thinner than about 0.040 inches in order to avoid delaying absorption rates and times. Adhesives, ultrasonic bonding or other attachment mechanisms may be used to connect the absorbent tip 202 to the holder tip 230, but are not preferred as they may alter the volume of fluid absorbed.

As used herein, the term “about” encompasses a variation of plus or minus 10%. While the above disclosure refers to absorbing various volumes of fluids within specified times, or less, one skilled in the art would understand that the larger end of the volume range cannot be absorbed within the minimum end of the time range. Thus, descriptions such as absorbing a specified range of blood in five seconds or less is to be construed rationally to encompass what may be practically achieved by the materials now available, and to the extent permitted by law while not invalidating the claims, construed to encompass what may be achievable by materials developed in the future.

The absorbent probe 18, 118 made of porous carbon is preferred. But the probe 18, 118 may be made of other materials, especially as the shapes of the probes 18, 118 are believed to be advantageous. The probes may thus be made from a variety of plastics such as polyethylene. Polyethylenes which may be employed include but are not limited to high density polyethylene (HDPE), low density polyethylene (LDPE) and ultra-high molecular weight polyethylene (UHMWPE). Plastic absorbent probes may also be made from polypropylene (PP), polyvinylidene fluoride (PVDF), polyamides, polyacrylates, polystyrene, polyacrylic nitrile (PAN), ethylene-vinyl acetate (EVA), polyesters, polycarbonates, or polytetrafluoroethylene (PTFE). Plastic holders and tips 230 may also be made from more than one of these plastics. Plastic probes 18, 118 made from about 30% polypropylene (PP) and about 70% polyethylene (PE) (wt:wt %) is believed suitable. In other embodiments when PP and PE are combined, PP may be present in a range of from about 100% to about 0% and PE may be present in a range of from about 0 to about 100% (100% to 0%:0% to 100% wt:wt %). When PE is combined with other polymers, the PE is present in at least about 50% (wt %). Plastic probes 18, 118 may also contain other additive materials, such as carbon, silica, control porous glass (CPG), ion exchange resins, modified silica, such as C-8 and C-18, or clays for improved binding and purification properties of the probes.

Porous probes made of are made from a variety of plastics fibers, such as continuous fibers or staple fibers are also believed suitable. Continuous fibers and staple fibers can be monocomponent fibers and/or bicomponent fibers. Examples of monocomponent fibers include, but are not limited to, glass, polyethylene (PE), polypropylene (PP), polyacrylate, polyacrylic nitrile (PAN), polyamides (Nylons), polyethylene terephthalate (PET), polybutylene terephthalate (PBT), copolyester (CoPET). Plastic fiber absorbent probes may further comprise cellulose based fibers such as cotton, rayon and Tencel. Examples of suitable bicomponent fibers include, but are not limited to, PE/PET, PP/PET, CoPET/PET, PE/Nylon, PP/Nylon, and Nylon-6,6/Nylon-6. The plastic-based probes 18, 118 may further comprise cellulose based fibers such as cotton, rayon and Tencel.

It is believed suitable to treat porous probes treated with polyelectrolyte solutions to increase hydrophilicity. In one embodiment, polyethyleneimine in aqueous or alcoholic solution may be applied to the probes. Polyelectrolytes are polymers with electric charges in the polymer chain. The polyelectrolytes that may be used in this application include: one or more of a surfactant, phosphate, polyethylenimine (PEI), poly(vinylimidazoline), quaterized polyacrylamide, polyvinylpyridine, poly(vinylpyrrolidone), polyvinylamines, polyallylamines, chitosan, polylysine, poly(acrylate trialkyl ammonia salt ester), cellulose, poly(acrylic acid) (PAA), polymethylacrylic acid, poly(styrenesulfuric acid), poly(vinylsulfonic acid), poly(toluene sulfuric acid), poly(methyl vinyl ether-alt-maleic acid), poly(glutamic acid), dextran sulfate, hyaluric acid, heparin, alginic acid, adipic acid, or chemical dye. It is also believed suitable to treat polyelectrolyte-treated probes with additional treatments, such as exposure to surfactant solutions or heparin.

Functional additives to the porous carbon and porous plastic absorbent probes are also believed to include, but are not limited to chelating agents, such as ethylene diaminetetraacetic acid (EDTA), surfactants, such as anionic surfactant, cationic surfactant or non-ionic surfactant, DNA stabilizing agents, such as uric acid or urate salt, or a weak acid, such as Tris(hydroxymethyl)aminomethane (TRIS). Functional additives are also believed to include but are not limited to a chaotropic agent, such as urea, thiourea, guanidinium chloride, or lithium perchlorate. Absorbent probes may also contain an anti-coagulant, such as heparin, citrate and/or chelating agents.

It is believed that suitable surfactants may be used with the carbon or plastic-based probes 18, 118, including an anionic surfactant, for example sodium dodecylsulfate (SDS), sodium dodecyl sulfate (SDS), sodium dodecyl benzenesulfonate, sodium lauryl sarcosinate, sodium di-bis-ethyl-hexyl sulfosuccinate, sodium lauryl sulfoacetate or sodium N-methyl-N-oleoyltaurate, a cationic surfactant, such as cetyltrimethylammonium bromide (CTAB) or lauryl dimethyl benzyl-ammonium chloride, a non-ionic surfactant, such as nonyl phenoxypolyethoxylethanol (NP-40), Tween-20, Triton-100 or a zwitterionic surfactant, such as 3-[(3-Cholamidopropyl)dimethylammonio]-1-propanesulfonate (CHAPS). Fluorosurfactants may also be used, such as Zonyl® fluorosurfactant from DuPont. It is believed that different surfactants can be combined together to obtain better hydrophilic results.

Thus, the probes 18, 118 may be made of various materials, including polyethylene, polypropylene, polyvinylidene fluoride, polyamide, polyacrylate, polyacrylic nitrile, ethylene-vinyl acetate, polyester, polycarbonate, polystyrene, polytetrafluoroethylene, or cellulose, or a combination thereof. Sintered probes made of these materials with an average pore size of 40 microns and pore volume of 38% are believed suitable. Such porous probes are described in U.S. Pat. No. 8,920,339, the complete contents of which are incorporated herein by reference. One method believed suitable for making a porous probe of plastic particles involves pyrolizing at a temperature ranging from about 200° F. to about 700° F., with a more preferred range being pyrolizing plastic particles at a temperature ranging from about 300° F. to about 500° F. The pyrolizing temperature depends on the plastic particles being pyrolized.

Plastic particles may be pyrolized for a time period ranging from about 30 seconds to about 30 minutes, and advantageously pyrolized for about 1 minute to about 15 minutes and more preferably for a time period ranging from about 5 minutes to about 10 minutes. The plastic pyrolizing process may include heating, soaking, and/or cooking cycles. Moreover the pyrolizing of plastic particles may occur under ambient pressure (1 atm) or under pressures greater than ambient pressure.

The holders and absorbent probes are provided in packages, sterilized. It is believed that suitable sterilization may be achieved using such techniques as gamma irradiation, plasma, ethylene oxide gas, dry heat or wet heat. The carbon probes 118 may be made using various carbon materials formed into the desired probe shape. Suitable processes are believed described in U.S. Pat. No. 8,383,703, the complete contents of which are incorporated herein by reference. This patent describes a process for producing solid beads of polymeric material such as a phenolic resin having a mesoporous structure. The process includes the steps of combining a stream of a polymerizable liquid precursor (e.g. a novolac and hexamine as cross-linking agent dissolved in a first polar organic liquid e.g. ethylene glycol with a stream of a liquid suspension medium which is a second non-polar organic liquid with which the liquid precursor is substantially or completely immiscible e.g. transformer oil containing a drying oil. The process also includes the step of mixing the combined stream to disperse the polymerizable liquid precursor as droplets in the suspension medium e.g. using an in-line static mixer. More specifically, the process includes forming a combined stream from a stream of a polymerizable liquid precursor and a stream of a liquid dispersion medium with which the liquid precursor is substantially or completely immiscible, and then treating the combined stream so as to disperse the polymerizable liquid precursor as droplets in the dispersion medium. The droplets are then allowed to polymerise in a laminar flow of the dispersion medium so as to form discrete solid beads that cannot agglomerate. Then the beads are removed from the dispersion medium. The dispersive treatment time is less than 5% of the laminar flow polymerization time so that agglomeration of the liquid precursor during dispersive treatment is substantially avoided. Preferably, the stream of polymerizable liquid precursor comprises polymerizable components in solution in a first polar organic liquid, and the liquid dispersion medium comprises a second non-polar organic liquid, with the first and second organic liquids being substantially immiscible.

Advantageously the process includes: forming the polymerizable liquid precursor by combining and mixing first and second component streams thereof in an in line mixer. Moreover, the first component stream preferably comprises a phenolic nucleophilic component dissolved in a pore former and the second component stream preferably comprises a cross-linking agent dissolved in the pore former, where the pore former is ethylene glycol, the phenolic nucleophilic component is a novolac with a molecular weight of less than 1500, and the cross-linking agent comprises hexamethylenetetramine, melamine or hydroxymethylated melamine.

The method of U.S. Pat. No. 8,383,703 also includes a method for carbonizing and activating carbonaceous material, which comprises supplying the material to an externally fired rotary kiln maintained at carbonizing and activating temperatures where the kiln has a downward slope to progress the material as it rotates. The kiln also has an atmosphere substantially free of oxygen provided by a counter-current of steam or carbon dioxide. Annular weirs may be provided at intervals along the kiln to control progress of the material. In this method, the carbonaceous material may include material of vegetable origin, but preferably comprises beads of phenolic resin.

The process further includes the step of allowing the droplets to polymerise in a laminar flow of the suspension medium so as to form discrete solid beads that cannot agglomerate, as well as the step of recovering the beads from the suspension medium.

Also provided is an apparatus for forming discrete solid beads of polymeric material. In other embodiments, a method is provided for carbonizing and activating carbonaceous material, and an externally fired rotary kiln for carbonizing and activating carbonaceous material.

It is believed that an advantageous process for making the absorbent probes 118 is to mill phenolic resin and classify the resulting milled particles. A sieve or mesh classifier is preferred to exclude large sized or large diameter particles and small sized particles. The resin is milled into particles that are on average 5 to 6 times the size of the pores desired in the probe 118. The carbon based probe is believed usable for biological purposes (especially blood) with average pore sizes as low as 10μ and as large as 150μ, with other pore sizes suitable for other applications. Average pore size may be determined by bubble point testing using air. The resin particles for a 40μ pore size preferably have a one sigma distribution with 65% of the resin sized between about 160μ and 240μ for a 200μ resin particle. Similarly, for a 150μ pore size the resin preferably has about 65% of the resin sized between 750-1080μ, using sieve classification to size the particles

The classified resin may be mixed with a pore former such as ethylene glycol and may be combined through extrusion or in a tablet press machine which machines are known in the art. If extruded the extruded shape may be cut to the desired length to form probes 118 or pressed to the desired shape to form probes 118 with the extruded pieces being formed in tablet press machines to the desired shape.

One resin believed suitable is described in U.S. Pat. No. 8,227,518, the complete contents of which are incorporated herein by reference. The patent describes a cured porous phenolic resin that can be made by cross-linking a phenol-formaldehyde pre-polymer in the presence of a pore former, preferably ethylene glycol. This patent describes a method of making mesoporous carbon beads including the following steps: (a) providing a nucleophilic component which comprises a phenolic compound or a phenol condensation prepolymer optionally with one or more modifying reagents selected from hydroquinone, resorcinol, urea, aromatic amines and heteroaromatic amines; (b) dissolving the nucleophilic component in a pore former selected from the group consisting of a diol, a diol ether, a cyclic ester, a substituted cyclic ester, a substituted linear amide, a substituted cyclic amide, an amino alcohol and a mixture of any of the above with water, together with at least one electrophilic cross-linking agent selected from the group consisting of formaldehyde, paraformaldehyde, furfural and hexamethylene tetramine; (c) dispersing the resulting solution into a mineral oil to form beads; (d) condensing the nucleophilic component and the electrophilic cross-linking agent in the presence of the pore former while dispersed in the mineral oil to form beads of porous resin; (e) removing the beads of porous resin from the mineral oil; and (f) carbonizing the beads of porous resin to form mesoporous carbon beads by treatment in an inert atmosphere at about 600° C. to about 800° C. with a heating rate up to about 10° C. per minute of treatment time.

That method also preferably includes a nucleophilic component that is a phenol-formaldehyde novolac, with the modifying reagent comprising aniline, melamine or hydroxymethylated melamine. These preferred variations also may advantageously incorporate dispersed heteroatoms into the porous resin and may further incorporate metal heteroatoms into the resin by dissolving a metal salt in the pore former. Further, the method may include incorporating non-metal heteroatoms into the resin by adding an organic precursor containing the heteroatoms to the pore former.

The basic method of U.S. Pat. No. 8,227,518 also preferably uses a pore former comprising ethylene glycol, or alternatively a pore former is selected from the group consisting of 1,4-butylene glycol, diethylene glycol, triethylene glycol, .gamma.-butyrolactone, propylene carbonate, dimethylformamide, N-methyl-2-pyrrolidone and monoethanolamine. Advantageously, at least 120 parts by weight of the pore former are used to dissolve 100 parts by weight of the nucleophilic component. Further, there is dissolved in the pore former as electrophilic cross linking agent hexamethylene tetramine at a concentration of at least 9 parts by weight per 100 parts by weight of the nucleophilic component. This resin production method also advantageously uses a nucleophilic component which is a phenol-formaldehyde novolac and an electrophilic cross-linking agent which is hexamine are dissolved in the pore former which is ethylene glycol by smoothly increasing the temperature to 100-105° C., with the resulting solution dispersed in the mineral oil at about the same temperature, and with the temperature gradually raised to 150-160° C. to complete cross-linking of the resin.

The resin made using the process of U.S. Pat. No. 8,227,518 may be formed by combining a nucleophilic component and an electrophilic crosslinking agent. The method makes a solid cured porous phenolic resin having mesopores/macropores of diameter greater than 2 nm as estimated by nitrogen adsorption porosimetry. The resin may have a differential of pore volume V with respect to the logarithm of pore radius R (dV/d log R) for pores of size 2-10 nm being less than values of dV/d log R for pores of size >10 nm, and values of dV/d log R being >0.2 for at least some values of pore size in the range 10-50 nm. The method includes reacting without catalyst in a pore-forming solvent: (a) a nucleophilic component and (b) an electrophilic crosslinking agent, with an amount of pore former exceeding the capacity of the cross-linked resin domains. The method also includes forming a solution with partially cross-linked polymer between the domains and increasing the volume of material in the voids between the domains and thereby giving rise to mesoporosity. The solvent is selected from the group consisting of diols, diol-ethers, cyclic esters, linear and cyclic substituted amides, aminoalcohols and optionally added water. The nucleophilic component includes a phenol condensation prepolymer optionally with one or more modifying reagents selected from the group consisting of hydroquinone, resorcinol, urea, aromatic amines and heteroaromatic amines. The electrophilic crosslinking agent is selected from the group consisting of formaldehyde, paraformaldehyde, furfural, hexamethylene tetramine, melamine and hydroxymethylated melamine. The pore-forming solvent preferably includes ethylene glycol while the nucleophilic component preferably includes novolac and the cross-linking agent includes hexamine and the pore former is in an amount >120 parts by weight per 100 parts by weight of novolac. It is believed advantageous if about 9 parts by weight of hexamine are used per 100 parts by weight of novolak. The resulting resin may advantageously be produced in the form of beads or powder. Advantageously the process uses a solution of the novolak and hexamine in ethylene glycol that is smoothly increased in temperature to about 100-105 degrees Centigrade and dispersed into oil at about the same temperature after which the temperature is gradually raised to about 150-160 degrees Centigrade to complete cross-linking. The resin may be produced in the form of powder with particles between 1 and 1000 μm, or in the form of beads of about 5-2000 μm. The method may also include removing the pore former below 100 degree Centigrade by washing the resin with water or by vacuum distillation.

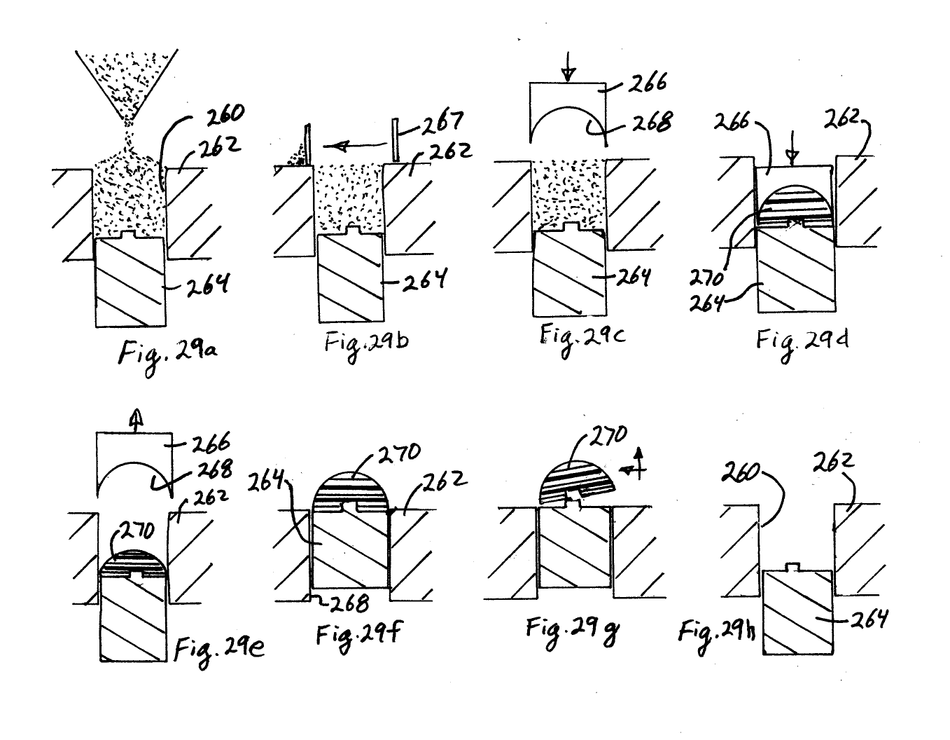

The preferred way to make the absorbent probe is believed to be pressing the resin into shape by powder compaction to form a probe-blank 270 and then pyrolize it that probe-blank after which the pyrolized probe undergoes a process to increase its wettability or hydrophilic properties. Referring to FIGS. 21a-21c and 29a-h, a pellitizing manufacturing is shown. In FIG. 29a, the resin material, preferably in powder or small bead form, is placed into a cavity 260 in die 262, usually from above as the die typically has a closed ended bottom with FIG. 29a shown the bottom closed by a bottom punch 264. Preferably the die has an annular cavity 260 with a movable bottom punch 264 and a movable top punch 266, each of which is selected to produce a probe 18, 118 of the desired shape, preferably a shape similar to that shown in FIG. 21d, 22, 26 or 28. For probes 118 with a cavity 126 to receive a mounting post, the upper die must have a male projection shaped to produce the desired cavity 126. The die 262 is preferably used with a pharmaceutical press which mixes and compresses precise quantities of materials.

The volume or mass of the powder or material placed in the die cavity 260 is determined by the position of the lower punch 264 in the die 262, the cross-sectional area of the die 262 and the resin density. Adjustments in the total weight or mass are preferably made by repositioning the lower punch 264. As seen in FIGS. 29a-29b, the resin is added to the top of the die 262 and the cavity 260 in the die 262 is filled to a predetermined level, volume or mass. The material in the die may be leveled off (FIG. 29b) by a scraper 267 or air or other means if the die volume is used to determine the amount of material used. After filling die 262, the upper punch or top punch 266 is lowered into the die to mate with the die cavity 260 surrounded by the annular sides of the die. The upper punch has a die cavity 268 configured to form the basic desired shape of probe 18, 118 before pyrolizing. Both the top and bottom punches 264, 266 and die 262 cooperate to form the shape of the probe before pyrolizing.

Referring to FIG. 26c-29e, the resin in the die 262 is compressed, typically in one or two stages. A two stage compression may use a pre-compression or tamping, followed by a main compression whereas a single stage omits any pre-compression step. A controlled amount of resin is compressed to a selected geometry. The mass of resin added to the die 262 is inversely correlated to the porous volume of the compressed resin. The mass added to the die 262 is chosen so that the carbon derived from the resin has a porous volume that is preferably between 20-40%. The porous volume can be determined by wicking fluid into the final part to determine the amount of water (or other solvent) absorbed into the part. For commercial production the compression occurs fast, 50-500 ms per probe.

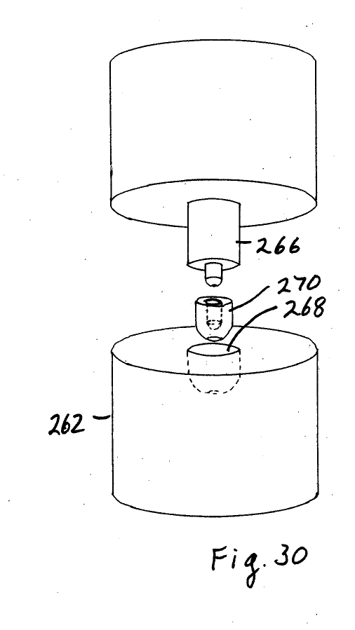

Referring to FIGS. 29f to 29h, following compression, the top punch 266 is removed from the die 262 and cavity 260 to decompress the die-shaped probe-blank 270. The formed probe-blank 270 may be removed by inverting the die 262 or the probe-blank may be ejected from the die 262 by raising the lower punch 264 to eject the formed probe-blank, preferably by lifting the lower die until the upper surface of the bottom punch 264 is flush with the top face of the die 262. Physical grippers, gravity or gas ejection may be used to remove the probe blank 270. Preferably, directed jets of clean air or inert gas jets remove the probes and provide some cooling and direction to the ejected probe-blanks 270 formed by the die 262 so the blanks may be collected for further processing. The sequence is repeated for subsequent probe-blanks, with the bottom punch 264 being lowered and ready to receive material as shown in FIG. 29h. Further processing steps include inserting a post 120 into the probe-blank 290, preferably by inserting the post into a mating recess formed in the probe-blank, and then pyrolizing the probe. The probe may be pyrolized before inserting the post 120. The top and bottom punches 266, 264 may have their configurations and functions varied as shown in FIG. 30, in which the top punch 266 has a stepped boss configured to fit into cavity 260 and form a recess for post 130 in the bottom of the probe blank 270.

The volume or mass of material placed into the die cavity 260 is controlled because fluctuations in the weight or mass that is compressed will vary the probe-blank 270 porosity and ultimate porosity of the completed probe. Thus, a uniform size and density of material is desired, as is an even or uniform flow of material into the die cavity 260 is desired in order to allow a fixed flow time to introduce a predetermined mass of material into the die cavity 260. Advantageously, the material is in powder form or the form of small beads of predetermined size as described above in order to more easily and uniformly achieve a consistent mass in the die cavity 260 and a more uniform porosity in the compressed probe-blank. The powder or bead material also advantageously has a consistent viscosity sufficient to avoid sticking to the die tooling which may arise from inadequate lubrication of the die parts, or from worn or dirty tooling, or from a sticky powder or bead material. Finally, if the carbon resin is formed at too high of a temperature the resulting powder and beads of carbon resin may be brittle from over-curing, and that may impede the formation of a suitable probe-blank 270.

Additional concerns arising during forming the probe-blank 270 are capping, lamination or chipping which are caused by a sufficiently large pocket of air compressed during the probe-blank formation which pocket of air is heated during compression that forms the probe-blank with the pocket of heated air expanding when the punch is released. This may cause fragmentation from air expansion when the forming pressure is released by removal of the punch and may cause localized heating or over-curing during formation of the probe-blank 270 which may render portions of the probe-blank brittle and more likely to fracture or chip. Thus, uniform particle sizing, distribution and bulk density are desired to reduce these concerns. As moisture can also similar problems, the moisture content is also preferably controlled. Thus, the process includes controlling one or more of the brittleness, pore former, composition, moisture content, lubricity, viscosity, bulk density and the particle size and distribution of the material introduced into the die cavity 260 to form the probe-blank 270, and by controlling the compression used to form the probe-blank.

After the probe-blanks 270 are formed they are carbonized by pyrolysis (i.e., in the absence of oxygen) at about 600° C. to about 800° C. The resulting shape of the pyrolized probe-blank is smaller than the probe-blank 270 before pyrolysis. The resulting shape after pyrolysis varies with the time and temperature of pyrolysis and the formulation of the material used to form the probe-blank, especially including one or more of the particle size, amount of pore former, the relative amount of compression in the die 262 and the absolute amount of compression force used in forming the probe-blank 270. At this stage of manufacturing the pyrolized probe is wetable by extended exposure to water and may take 2-5 minutes to wet the surface and portions of the interior of the pyrolized probe.

A high temperature pyrolysis step may be performed after the lower-temperature pyrolysis step. After the pyrolysis step at 600-800° C. it is believed that the carbon in the probes may optionally be further activated by a higher temperature pyrolysis step by increasing the temperature to about 900° C., preferably but optionally without any intervening cooling step, for further pyrolysis to increase the surface area of the carbon and to thereby increase the amount of molecules that may interact with that increased surface area of the probe. Advantageously this optional higher temperature pyrolysis step follows without delay the initial pyrolysis step at 600-800° C. and without cooling the pyrolized probes to a lower temperature below that used for pyrolysis.

A low temperature, heat sterilization step may optionally be performed after the pyrolysis step. After the pyrolysis step (and after any optional high temperature pyrolysis) the pyrolized carbon probe is optionally, but preferably sterilized by heating the probe between about 250° C. and about 350° C., preferably in the presence of pure oxygen and less preferably in the presence of air. If this temperature sterilization step is performed in the presence of oxygen then it may add oxygen moieties to the carbon surface, typically alcohols and carboxylic acids. These oxygen moieties may create a negative charge which may render the surface more wettable or hydrophilic. Sterilization in the presence of air induces the possibility of forming other moieties which may reduce the hydrophilic properties.

A plasma treatment step may optionally, but preferably be performed after the lower-temperature pyrolysis step. Treatment with an oxygen plasma or treatment with plasma enhanced CVD may be used to increase wettability and increase hydrophilic properties. A treatment by oxygen plasma or PCVD is believed sufficient to increase hydrophilic properties. It is believed desirable to use, only one of the high temperature pyrolysis, heat sterilization, oxygen plasma or oxygen PCVD processes to increase the hydrophilic properties of the pyrolized carbon absorbent probe 118, but combinations of these processes are also believed suitable.

The pressing or compaction of the carbon resin, pore former and selected materials to form the probe-blank 270 is believed preferable because the uniformity of the mass loading into the die cavity 260 results in a porous probe 118 with a uniform pore spacing throughout the volume of the carbon probe 118 and does so consistently so there is little part to part variation. The pyrolysis process results in a stable absorbent probe 118 and the subsequent steps to increase the hydrophilicity of that probe 118 do not reduce that stability. Moreover, the thermal stability of a carbon absorbent probe 118 is very high and provides a desirable support for several analytical conditions such as vacuum or high temperature as may arise in a mass spectrometer. Moreover, the probe offers the ability to absorb a predetermined quantity of fluid within a specified time that can be very short, measured in second.

The absorbent probe 118 is dark in color, usually black as it is made of pyrolized carbon. The black color may make it more difficult to visually determine when the probe 118 is full of the absorbed fluid. Suitable visual indicators to reflect when the absorbent probe 118 is at its capacity may be provided. Illustrative visual indicators include an indicator strip in fluid indication with the absorbent probe to change color when the probe 118 is full. For example, a disk of porous paper at the base or upward end of the absorbent probe opposite the distal tip or end of the probe, which disk is white or another light color to display the color of the absorbed fluid when it passes through the probe to contact the disk. For example, a white disk could turn red when an absorbent tip 118 has reached it designed capacity of absorbed blood, with a portion of the blood passing into and preferably throughout the disk to provide a visual indication that the blood has reached the location of the disk and/or that the probe is full.

Alternatively, a small capillary tube may extend from the part of the probe that is most likely to fill up last with the absorbed fluid, typically the base of the probe. The small capillary wicks the absorbed fluid rapidly along the capillary to provide a more visible indicator that the probe 118 is full.

Advantageously, the indicator displays the color of the absorbed fluid but alternatively, the indicator may be treated with a chemical that reacts with the intended absorbed fluid(s) to change color and if so, the chemical is advantageously selected so it does not detrimentally affect the planned analysis of the absorbed fluid.

The absorbent probe may contain additives that are preferably added in a liquid form and then dried onto the probe 18, 118, with the additives being selected to interact beneficially with the liquid absorbed by the probe. It is believed that anticoagulants such as EDTA or Heparin could be added to the probe 18, 118 to prevent coagulation of blood once a sample has been collected. EDTA is believed to act as a sequestering agent that may deactivate metal ions and reduce the oxidation of fatty molecules and useful in applications where those aspects are advantageous. Gallic acid is believed suitable as an antioxidant and oxygen scavenger. Ascorbic acid is also believed suitable as an antioxidant. Surfactants such as SDS (sodium dodecyl sulfate) can be added to the probe 18, 118, preferably added in the liquid stage and then dried. The surfactants are believed useful for their soap-like properties (allows easier ablation of dried blood from the surface), and/or their ability to facilitate cell lysis. Cell lysis, the destruction or degradation of cells, is a critical feature of many genomics tests or oligonucleotide analysis because the oligonucleotides must be freed from the cell in order to be detected. Antioxidants such as sulfites, preferably potassium sulfite or sodium metabisulfite or butylated hydroxytoluene or tert-Butylhydroquione are believed useful to add to the probe, again preferably added in the liquid form and then dried. These materials are believed useful to act as antioxidants, disinfectants, and preservatives and are generally used to increase the stability of analaytes in the dried form once a sample has been collected. The tert-Butylhydroquione and butylated hydroxytoluene are believed useful in suppressing the formation of organic peroxides. Enzymes such as trypsin or beta-glucuronidase can be added to the probe 18, 118, again preferably added in the liquid form and then dried. These enzymes are typically used to modify or process the sample prior to drying in order to eliminate downstream processing. For example, Trypsin will digest an intact protein into known peptide fragments for detection while Beta-glucoronidase will convert a glucuronide back into its original form (i.e. Morphine Glucornide Morphine).

Once dried blood is reconstituted from the probe 18, 118 it may be treated in analytical workflows in a manner similar to whole blood or plasma. Likewise, once dried biological fluid is reconstituted from probe 18, 118 it may be treated in analytical workflows in a manner similar to the whole biological fluid. Thus, the carbon-based probes 18, 118 is believed suitable for use in performing numerous analysis, substituting for existing absorbent materials holding existing fluid samples of various biological fluids or other fluid samples in order to provide a more consistent absorption of fluid (especially liquid) in a shorter time and resulting in more accurate analysis, especially of the reconstituted dried samples. The following uses and analysis are prophetic. One test where the probe 18, 118 may be advantageously used is in testing for testosterone in a test sample. The method of using probe 18, 118 are believed to advantageously include removing the sample from the probe and then ionizing all or a portion of the testosterone present in the sample to produce one or more testosterone ions that are detectable in a mass spectrometer. All or a portion of the testosterone present in the sample is ionized to produce one or more testosterone ions, which may be isolated and fragmented to produce precursor ions. A separately detectable internal testosterone standard can be provided in the sample. In a preferred embodiment, the reference is 2,2,4,6,6-d₅ testosterone. The method for determining the presence or amount of testosterone in a test sample is believed to advantageously include the steps of: (a) purifying testosterone from the test sample by chromatography; (b) ionizing the purified testosterone to produce one or more testosterone ions detectable by a mass spectrometer having a mass/charge ratio selected from the group consisting of 289.1±0.5, 109.2.±0.0.5, and 96.9±0.5; and (c) detecting the presence or amount of the testosterone ion(s) by a mass spectrometer, wherein the presence or amount of the testosterone ion(s) is related to the presence or amount of testosterone in the test sample, wherein purification is achieved using a chromatography system which is connected in-line to the mass spectrometer.

The probes may also be used in a method for determining the presence or amount of testosterone in a test sample which method is believed to advantageously include obtaining a sample from the probe 18, 118 on which the sample was obtained and then: ionizing all or a portion of the testosterone present in the sample to produce one or more testosterone ions that are detectable in a mass spectrometer. Advantageously, all or a portion of the testosterone present in the sample is ionized to produce one or more testosterone ions, which may be isolated and fragmented to produce precursor ions. A separately detectable internal testosterone standard can be provided in the sample. In a preferred embodiment, the reference is 2,2,4,6,6-d₅ testosterone.

In more detail, the method for determining the presence or amount of testosterone in a test sample extracted from probe 18, 118, includes: (a) purifying testosterone from the test sample by high turbulence liquid chromatography (HTLC); (b) ionizing the purified testosterone to produce one or more testosterone ions detectable by mass spectrometry having a mass/charge ratio selected from the group consisting of 289.1±0.5, 109.2±0.5, and 96.9±0.5; and (c) detecting the presence or amount of the testosterone ion(s) by mass spectrometry, wherein the presence or amount of the testosterone ion(s) is related to the presence or amount of testosterone in the test sample. A method for determining the presence or amount of testosterone in a test sample is also believed to include obtaining a sample from the probe 18, 118 on which the sample was dried and then: (a) purifying testosterone from the test sample by turbulent flow chromatography; (b) ionizing the purified testosterone to produce one or more testosterone ions detectable by a mass spectrometer having a mass/charge ratio selected from the group consisting of 289.1±0.5, 109.2±0.5, and 96.9±0.5; and (c) detecting the presence or amount of the testosterone ion(s) by a mass spectrometer, wherein the presence or amount of the testosterone ion(s) is related to the presence or amount of testosterone in the test sample 0.5, 109.2±0.5, and 96.9±0.5; and (c) detecting the presence or amount of the testosterone ion(s) by a mass spectrometer, wherein the presence or amount of the testosterone ion(s) is related to the presence or amount of testosterone in the test sample. The analyzed samples may be obtained from urine, blood, serum or blood plasma absorbed onto probe 18, 118, dried, extracted and then analyzed using the described method(s). More details on these testosterone methods are disclosed in U.S. Pat. No. 6,977,143, the complete contents of which are incorporated herein by reference.

The probes 18, 118 may also be advantageously used in a method for determining vitamin D metabolites by mass spectrometry. One method believed to be faster and more accurate is a method for determining the presence or amount of 25-hydroxyvitamin D₃ in a sample by tandem mass spectrometry. The method is believed to include extracting the sample from the probe 18, 118 and then: (a) generating a protonated and dehydrated precursor ion of 25-hydroxyvitamin D₃ with a mass to charge ratio (m/z) of 383.16±0.5; (b) generating one or more fragment ions of the precursor ion; and (c) detecting the presence or amount of one or more of the ions generated in step (a) or (b) or both and relating the detected ions to the presence or amount of 25-hydroxyvitamin D₃ in the sample. The sample may be subjected to a purification step before ionization, or chromatography may be used to purify the sample before ionization.

Another method believed to be faster and more accurate for determining or amount of two or more vitamin D metabolites in a sample in a single assay, where the methods generally comprise ionizing a vitamin D metabolite in a sample and detecting the amount of the ion to determine the presence or amount of the vitamin D metabolite in the sample. A similar method may detect the presence or amount of two or more vitamin D metabolites in a single assay. More specifically, the methods are believed to include extracting the sample from the probe 18, 118 and then (a) ionizing the two or more vitamin D metabolites, if present in the sample, to generate protonated and dehydrated precursor ions specific for each of the two or more vitamin D metabolites; (b) generating one or more fragment ions of each of the precursor ions; and (c) detecting the presence or amount of one or more of the ions generated in step (a) or (b) or both and relating the detected ions to the presence or amount of the two or more vitamin D metabolites in the sample, and wherein the two or more vitamin D metabolites comprise 25-hydroxyvitamin D₃ and 25-hydroxyvitamin D₂, and wherein the precursor ion of 25-hydroxyvitamin D₃ has a mass/charge ratio (m/z) of 383.16±0.5 and the precursor ion of 25-hydroxyvitamin D₂ has a mass/charge ratio (m/z) of 395.30±0.5.

Another method believed to be faster and more accurate for determining or amount of 25-hydroxyvitamin D₂ in a sample by tandem mass spectrometry, where the method is believed to include extracting the sample from the probe 18, 118 and then: (a) generating a protonated and dehydrated precursor ion of the 25-hydroxyvitamin D₂ with a mass to charge ratio (m/z) of 395.30±0.5; (b) generating one or more fragment ions of the precursor ion; and (c) detecting the presence or amount of one or more of the ions generated in step (a) or (b) or both and relating the detected ions to the presence or amount of the 25-hydroxyvitamin D₂ in the sample. More details on these methods relating to analyzing vitamin D metabolites are described in U.S. Pat. No. 7,972,867, the complete contents of which are incorporated herein by reference.

Another method believed to be faster and more accurate for measuring the amount of a vitamin B2 in a sample uses samples obtained from probes 18, 118 with mass spectrometric methods for detecting and quantifying vitamin B2 in the sample utilizing on-line extraction methods coupled with tandem mass spectrometric techniques. On method believed suitable for determining the amount of vitamin B2 in a biological sample from a human includes obtaining a sample from probe 18, 118 and then: (a) adding an internal standard to the sample; (b) subjecting the sample to liquid chromatography; (c) ionizing vitamin B2 and the internal standard under conditions suitable to produce one or more ions detectable by tandem mass spectrometry; (d) determining the amount of the one or more ions by tandem mass spectrometry; and (e) comparing the amount of the one or more ions of vitamin B2 and the one or more ions of the internal standard to determine the amount of vitamin B2 in the sample.

Another method for determining the amount of vitamin B2 in a biological sample from a human that is believed suitable for use with a sample extracted from probe 18, 118 includes: (a) adding an internal standard to the sample; (b) precipitating protein from the sample; (c) subjecting the sample to liquid chromatography; (c) ionizing vitamin B2 and the internal standard under conditions suitable to produce one or more ions detectable by tandem mass spectrometry; (d) determining the amount of said one or more ions by tandem mass spectrometry; and (e) comparing the amount of said one or more ions of vitamin B2 and said one or more ions of the internal standard to determine the amount of vitamin B2 in the sample.

Another method for determining the amount of vitamin B2 in a biological sample from a human that is believed suitable for use with a sample extracted from probe 18, 118 includes: a. subjecting the sample, purified by mixed-mode turbulent flow liquid chromatography (TFLC) and high performance liquid chromatography (HPLC), to ionization under conditions suitable to produce one or more ions detectable by mass spectrometry; b. determining the amount of said one or more ions by tandem mass spectrometry; and c. using the amount of said one or more ions to determine the amount of vitamin B2 in the sample. More details on these methods relating to analyzing vitamin B2 are described in U.S. Pat. No. 8,399,829, the complete contents of which are incorporated herein by reference.

A determination method for a serum metabolic marker for early diagnosis of diabetic nephropathy is also believed suitable for use with a sample extracted from probe 18, 118. The method utilizes analysis technique and method such as gas mass spectrometry and mass liquid spectrometry for quantitative determination of endogenous small molecule compounds in urine of patients with diabetes and diabetic nephropathy, with the urine sampled using probe 18, 118. A solvent such as methanol is used to extract the small molecules from the urine sample retained by probe 18, 118. The sample may be derivatized using trimethylsilyl trifluoroacetyl (MSTFA), and analyzed by gas chromatography, mass spectrometry (GC/TOF-MS), ultra-high performance liquid chromatography, and time of flight mass spectrometry (UPLC/TOF-MS). A relative concentration difference between endogenous small molecule compounds in a pathological group and a normal group is calculated and compared, so as to be applied to clinical early diagnosis of diabetic nephropathy. Compared with other existing clinical diagnosis indexes, the method has advantages of wide adaptation range, sensitivity, simple sampling and operation, and no harm on the body. The method is more suitable for screening of diabetic nephropathy in early stage with some uncertain physiological and biochemical indexes, so as to avoid missing of the best treatment time due to delayed diagnosis. This method is described in more detail in China Patent Application CN102901789

The invention relates to a liquid chromatography-tandem mass spectrometry detection method of common drugs in human blood. According to the method of the invention, an extraction method, a purification method, chromatographic conditions and mass spectral conditions of the thirty-one common drugs in the human blood are established. The method has a minimum detection limit of 1 to 2 ng/mL. When the concentration is from 2 to 100 ng/ml, the linearity is good with a related coefficient of 0.9771 to 0.9995 and an RSD of less than 13.2%. The method of the present invention has the advantages of strong pertinence, simple operation, fast detection speed, wide detection area, and easy popularization and application, can be used for fast detection of entry-exit inspection and quarantine departments, disease control centers and police departments, and positive result confirmation. The drugs can be detected through blood sampling, so a detection escape phenomenon which may appear when entry-exit inspection and quarantine systems guard passes at ports is prevented.

Another method for isolating and storing, nucleic acid from a sample containing nucleic acid, such as a cell sample or cell lysate, that is believed suitable for use with a sample extracted from probe 18, 118 includes: isolating a nucleic acid on a solid phase medium, which is then dried, and which can be stored efficiently, such as at room temperature, in columns, tubes, and microwell plates having a wide variety of filters and other solid phase media, for extended periods of time, including days, weeks, and months. Such a method for isolating and storing nucleic acid, includes: a. providing a solid phase medium; b. applying a sample (obtained from probe 18, 118) that includes cells containing nucleic acid to the solid phase medium; c. retaining the cells with the solid phase medium as a cellular retentate and removing contaminants; d. contacting the cellular retentate with a solution comprising a surfactant or detergent; e. lysing the cellular retentate to form a cell lysate while retaining the cell lysate in the medium, the cell lysate comprising the nucleic acid; f. drying the solid phase medium with the cell lysate comprising the nucleic acid; and g. storing the dried solid phase medium with the nucleic acid. Advantageously, before the drying step f, the solid phase medium with the nucleic acid is washed to remove contaminants while the nucleic acid is retained in the solid phase medium.

Another method for isolating and storing nucleic acid that is believed suitable for use with a sample extracted from probe 18, 118 includes: a. providing a solid phase medium; b. applying a sample comprising cells containing nucleic acid to the solid phase medium and concentrating the cells in the solid phase medium; c. retaining the concentrated cells with the solid phase medium as a concentrated cellular retentate and removing contaminants; d. contacting the concentrated cellular retentate with a solution includes a weak base, a chelating agent and an anionic surfactant or detergent; e. lysing the concentrated cellular retentate to form a cell lysate while retaining the cell lysate in the medium, the cell lysate comprising the nucleic acid; f. drying the solid phase medium with the cell lysate comprising the nucleic acid; g. storing the dried solid phase medium with the nucleic acid for at least one week; and h. eluting the nucleic acid from the solid phase medium.

A method for isolating and storing DNA that is believed suitable for use with a sample extracted from probe 18, 118 includes: a. providing a solid phase medium, wherein the solid phase medium comprises a filter comprising a plurality of fibers, wherein the fibers include one of glass or silica-based fibers, plastics-based fibers or nitrocellulose or cellulose-based fibers; b. applying a sample comprising cells containing DNA to the solid phase medium and concentrating the cells in the solid phase medium; c. retaining the concentrated cells with the solid phase medium as a concentrated cellular retentate and removing contaminants; d. contacting the concentrated cellular retentate with a solution that includes at least one of comprising: a weak base, a chelating agent and an anionic surfactant or detergent; e. lysing the concentrated cellular retentate to form a cell lysate while retaining the cell lysate in the medium, the cell lysate containing DNA; f. drying the solid phase medium with the cell lysate comprising the DNA; g. storing the dried solid phase medium with the DNA at a temperature of 5° C. to 40° C. for at least one week; h. heating the DNA with the solid phase medium to an elevated temperature of 65° C. to 125° C.; and i. eluting the DNA from the solid phase medium. More details on these methods relating to isolating and storing nucleic acid and DNA are described in U.S. Published Patent Application 2006/0094015, the complete contents of which are incorporated herein by reference.

A method for assessing risk of a neurodegenerative disease or disorder in a subject that is believed suitable for use with a sample extracted from probe 18, 118 includes comparing a level of anti-β-amyloid-42 (Aβ₄₂) antibody in a biological sample from a subject to a normal level, wherein a lower level in the biological sample from the subject indicates the risk of the disease or disorder. In a specific embodiment, the disease or disorder is Alzheimer's disease (AD, with the sample being extracted from probe 18, 118. One such method for assessing risk of Alzheimer's Disease in a subject includes: (a) determining the level of anti-β-amyloid-42 (Aβ42) antibody in a biological sample (obtained using probe 18, 118) selected from the group consisting of blood, serum, and plasma from a subject and (b) comparing the level of anti-Aβ42 antibody in the biological sample from the subject to a normal level determined from an average of the level of anti-Aβ42 antibody in a biological sample from a population consisting of age-matched normal subjects who do not show any symptoms of neurodegenerative disease or disorder associated with amyloidosis, wherein a statistically significantly lower level in the biological sample from the subject indicates the risk of Alzheimer's Disease. An immunoassay, preferably an enzyme-linked immunosorbent assay is used to determine the level of anti-Aβ₄₂ antibody in the biological sample.

A method for assessing risk of Alzheimer's Disease in a subject that is believed suitable for use with a sample extracted from probe 18, 118 includes (a) determining the level of anti-β-amyloid-42 (Aβ42) antibody in a biological sample selected from the group consisting of blood, serum, and plasma from a subject, wherein the subject does not exhibit symptoms of cognitive dysfunction or memory dysfunction; and (b), comparing the level of anti-Aβ42 antibody in the biological sample to a normal level determined from an average of the level of anti-Aβ42 antibody in a biological sample from a population consisting of age-matched normal subjects who do not show any symptoms associated with Alzheimer's Disease, wherein a statistically significantly lower level in the biological sample from the subject indicates the risk of Alzheimer's Disease. The subject is preferably from a family that has a member or members with familial Alzheimer's disease and preferably in his or her seventh or eighth decade of life.

A method for immunologically measuring apolipoprotein B with good sensitivity is believed suitable for use with a sample extracted from probe 18, 118. The method includes sampling blood by use of probe 18, 118, drying the blood, and eluting apolipoprotein into a solution containing a surfactant. The probe is immersed in an eluent having phosphate, NaCl and a surfactant dissolved therein and adjusted to a pH of about 7.3 and is left overnight at about 4° C. The solution obtained by this method is diluted by a solution having phosphate and NaCl dissolved therein and adjusted to a pH of about 7.3 to prepare a measuring specimen. Apolipoprotein in this specimen is immunologically measured using an anti-apolipoprotein B antibody. By this method, even when blood sampling amount is very small, a measured value well correlative to that from the serum of blood sampled in large amount is believed obtainable.

A method for detecting a HSPG2 or a fragment of cancer is believed suitable for use with a sample extracted from probe 18, 118. The sample contains a cell from the sample obtained from probe 18, 118 with an immune reagent or a conjugate where the immune reagent has a first scFv antibody fragment (26-29 kDa) that specifically binds to membrane protein HSPG2 (e.g., Perlecan). The immune reagent may further include a second scFv antibody fragment operably linked to the first scFv antibody fragment to form a diabody. The diabody is preferably about 52-60 kDa. The first and second antibody fragments are advantageously linked by means of a linker and more preferably linked by a peptide linker. The conjugate may include an immunoglobulin conjugated to a detection agent and/or therapeutic agent, where the detection agent or therapeutic agent may include a radionuclide. The radionuclide may be metallic. The conjugate may include a radionuclide. The detection agent may include a fluorescent group. The immune reagent may be conjugated to a therapeutic agent, such as a cytotoxic compound. The method may further include the step of measuring a signal from the detection agent, wherein a signal from the test sample that is greater than a signal from a non-cancerous control sample indicates the presence of cancer cells in the test tissue sample, and preferably with the signal from the test sample being 1-100% greater than the signal from the control sample.

The probes 18, 118 described herein are believed to be especially useful in numerous chemical separation methods to absorb fluids for later drying, shipping, reconstitution and/or analysis using existing chemical separation methods to extract analytes from the probe and/or to remove disadvantageous components of the biological matrix from the extract. The extract or purified extract is then analyzed by a variety of analytical methods. The probes and samples contained on the probes are believed usable for separation and processing methods such as solid phase extraction (SPE), protein precipitation methods using acid, salts, or organic solvents (including modifiers such as zinc sulfate or formic acid), liquid-liquid extraction (LLE) including solid supported liquid-liquid extraction, solid liquid extraction, gas chromatography (GC), liquid chromatography (LC), affinity chromatography, ion exchange chromatography, size exclusion chromatography, gel permeation chromatography, thin layer chromatography, and capillary electrophoresis.

The ability to absorb specific quantities of fluids in short times, the porosity allowing drying in conjunction with the non-reactive porous structure of the probe, and the reconstitution of the dried fluid are believed to make the probes 18, 118 described herein especially useful to provide samples suitable for analysis using existing processes, procedures and analytical instruments, including immunoassay analyzers and systems, enzyme linked immunosorbent assays, clinical chemistry analyzers, colorimetry (enzymatic assays or otherwise), mass spectrophotometers (including inductively coupled plasma mass spectrometry, TOF, and tandem mass spectrometry), UV-Visible spectrometers, flame and flameless atomic absorption spectrometers, fluorescence spectrometers, molecular absorption spectrophotometers, nuclear magnetic resonance analyzers, Fourier transform infrared spectrometers, X-ray diffraction analyzers, microscopes (transmission electron, scanning electron, atomic force, optical, and field emission scanning microscopes), gas chromatography (GC) used in conjunction with mass spectrophotometers or other detectors such as a UV-Vis, liquid chromatography (LC) used in conjunction with mass spectrophotometers or other detectors such as a UV-Vis, surface plasmon resonance, near IR and Raman analysis, DNA and RNA analyzers, polymerase chain reaction methods and lateral flow tests.

Because of the non-reactivity of the carbon-based probes 18, 118 the probes are believed suitable for use with processes and procedures to extract numerous analytes from the probes and/or from the fluids or reconstituted fluids obtained from the probes and for later analysis of those fluids, using existing processes. The probes 18, 118 are believed especially useful with fluids and dried fluids containing such analytes as: acylcarnitines, alcohols and alcohol metabolites such as phosphatidylethanol (a marker for chronic alcohol use), amphetamines (e.g., amphetamine, ephedrine, MDA, etc.), amino acids. anticoagulant poisons (e.g., Brodifacoum; Bromadiolone; Chlorophacinone), anti-depressants (e.g., Desmethyltrimipramine, Doxepin, Fluoxetine, etc.), antiepileptics (e.g., Clonazepam, Phenytoin, Sodium Valproate), appetite suppressants (e.g., Leptin), barbiturates (e.g., Amobarbital; Butabarbital; Butalbital; Pentobarbital), bath Salts (MDPV; Mephedrone; Methoxetamine), benzodiazepines (e.g., Diazepam; Estazolam; Flurazepam), beta-blockers (e.g., Acebutolol; Atenolol; Labetalol; Metoprolol), cannabinoids (e.g., 11-Hydroxy Delta-9 THC; Delta-9 Carboxy THC; Delta-9 THC), carotenoids (e.g., Lutein, Zeaxanthin), total cholesterol, HDL, LDL and triglycerides, cocaine and degradation products (e.g., Benzoylecgonine; Cocaethylene; Cocaine; Ecgonine Methyl Ester), diabetes markers and risk factors (e.g., Glucose, HbA1c, glycated albumin, Adiponectin, etc.), ephedrines (e.g., Ephedrine; Methylephedrine; Norpseudoephedrine), fatty acids, flurocarbons (e.g., chlorodifluoromethane; trichlorotrifluoroethane), glycols (e.g., diethylene glycol; ethylene glycol), herbicides (e.g., 2,4,5-Trichlorophenoxyacetic Acid; 2,4-D; Dicamba), hormones (e.g., Steroids, Testosterone, Progestogens, Thyroxine, Cortisol etc.), hypoglycemic agents (e.g., pioglitazone; repaglinide), immunosuppressants (e.g., Tacrolimus, Cyclosporin A, Sirolimus), inflammation markers (e.g., homocysteine), metals (e.g., lead, iron, arsenic, mercury zinc, etc.), nonsteroidal anti-inflammatory drugs (e.g., Etodolac; Fenoprofen; Flurbiprofen; Ibuprofen), oligonucleotides (e.g., DNA, RNA, and sequencing approaches), Omega-3, Omega-6, steroids, substituted cathinones (e.g., 1-(1,3-benzodioxol-5-yl)-2-(ethylamino)propan-1-one; 1-(1,3-benzodioxol-5-yl)-2-(methylamino)butan-1-one), vitamins (e.g., vitamin A, C, D, B2, B12, E, etc.), proteins (antibodies (e.g., IgG, IgM, etc.), enyzymes (along with all major classes including defensive, structural, transport and receptor proteins like Insulin growth factor-1, Transferrin receptor), glycated hemoglobin and food allergens) and peptides (markers for proteins, diabetes, cardiac or other biological markers).

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

The above description is given by way of example, and not limitation. Given the above disclosure, one skilled in the art could devise variations that are within the scope and spirit of the invention, including various ways of enclosing the device 10 or holder 114 in a protective case 10, and various ways of configuring the sample end 12 or probe 114. Moreover, while the preferred use of the holder 114 and probe 18, 118 is to absorb blood, its use is not so limited as the method and apparatus disclosed herein may be used to absorb, dry and transport other fluids. Further, the various features of this invention can be used alone, or in varying combinations with each other and are not intended to be limited to the specific combination described herein. Moreover, while the above described method and apparatus is preferably used to sample and test human blood, it may be used to sample and test blood from any animal. Moreover, the method and apparatus may be used to sample and test human and animal bodily fluids other than blood, and may further be used to sample and test any fluid. Thus, the invention is not to be limited by the illustrated embodiments.

What is claimed is:

1. A biological fluid sampling device, comprising:
an absorbent probe made of an open cell, porous carbonized material; and
a holder connected to the probe and configured to allow a user to manually manipulate the holder and probe during use.

2. The device of claim 1, wherein the absorbent probe is of sufficient size to absorb for analysis about 1 μl to about 100 μl of blood in about 2-5 seconds without separating the blood from plasma, the probe having a length of less than about 5 mm and a cross-sectional area of less than about 20 mm² with a majority of the exterior surface of the probe being exposed and available for placing against a fluid sample on a surface to absorb the sample;

3. The device of claim 2, wherein the probe is made of pyrolyzed carbon with about 90% or more of the absorbent probe made of carbon, by weight.

4. The device of claim 2, wherein the probe is made of pyrolyzed carbon with about 95% or more of the absorbent probe made of carbon, by weight.

5. The device of claim 3, wherein the holder is dimensionally suited for use with devices that can manipulate a pipette tip.

6. The device of claim 2, wherein the holder is disposable and the probe has a pore volume of about 35% or more.

7. The device of claim 3, wherein the size is sufficient to absorb about 20 microliters.

8. The device of claim 3, wherein the probe has a cylindrical portion that is sized to fit into a mating opening in the holder to connect the holder to the probe.

9. The device of claim 3, wherein the probe and holder are sterile and packaged in a sterile container.

10. The device of claim 2, wherein the probe contains dried anti-coagulant.

11. The device of claim 3, wherein the probe contains dried anti-coagulant.

12. The device of claim 2, wherein the probe contains at least one of a reference standard, a dried stabilizer or a modifier.

13. The device of claim 2, wherein the probe contains dried blood.

14. The device of claim 3, wherein the probe contains dried blood.

15. The device of claim 3, further comprising a container having a recess configured to enclose the holder for transportation of the holder.

16. The device of claim 15, wherein the container has a plurality of openings allowing air to access the probe.

17. A process for use in testing a blood sample, comprising:
placing an absorbent probe in physical contact with a blood sample, the absorbent probe being made of a carbonized, porous carbon material and connected to a holder, the absorbent probe being configured to absorb a predetermined maximum volume of blood of about 1 μl to about 100 μl, the absorbent probe having a majority of its exterior surface exposed and accessible for absorption of blood from a surface;
maintaining a portion of the exterior surface of the probe in contact with the blood sample until the predetermined amount of blood is absorbed by the probe;
removing the probe from contact with the blood; and
drying the blood in the probe without contaminating the blood.

18. The process of claim 17, wherein the absorbent probe is made of pyrolyzed carbon with about 90% or more of the absorbent probe made of carbon.

19. The process of claim 17, wherein the absorbent probe is made of pyrolyzed carbon with about 95% or more of the absorbent probe made of carbon, by weight.

20. The process of claim 18, wherein the predetermined time is less than about five seconds.

21. The process of claim 18, wherein the blood sample is on a live animal when contacted by the probe.

22. The process of claim 18, wherein the probe is configured to absorb a predetermined amount of blood of about 5-15 microliters.

23. The process of claim 18, wherein said majority of the exterior surface of the probe has a porosity of about 30% to 50%.

24. The process of claim 18, further comprising placing the probe with the dried blood in a compartment within a container.

25. The process of claim 18, further comprising placing the probe with dried blood in a container along with a fluid selected to reconstitute the dried blood in the probe.

26. The process of claim 18, comprising a plurality of probes each held in a pipette tip and each containing dried blood, the pipette tips being held in a tray.

27. A kit for collecting body fluids, comprising:
a plurality of holders each having a manipulating end and opposite thereto an absorbent probe made of a hydrophilic, porous carbon material configured to absorb a predetermined volume of about 1 μl to about 100 μl of blood within about 1-5 seconds, the probe having a majority of its exterior surface exposed for potential use in absorbing the blood;
a container having a plurality of compartments, each configured to releasably receive a different one of the holders and its probe, the container and holder configured to prevent the probes from abutting the compartment within which the holder and probe are placed, the container having openings in each compartment to allow air to enter each of the compartments and reach the probe within the compartment with which the openings are associated.

28. The kit of claim 27, wherein the absorbent probe is made of pyrolyzed carbon with about 90% or more of the absorbent probe made of carbon, by weight.

29. The kit of claim 27, wherein the absorbent probe is made of pyrolyzed carbon with about 95% or more of the absorbent probe made of carbon, by weight.

30. The kit of claim 28, further comprising a plurality of access ports with each port associated with a different one of the compartments, each port located to allow printing onto the manipulating end of the holder in the compartment with which the port is associated.

31. The kit of claim 28, wherein the container has two parts cooperating to form tubular compartments containing different ones of the absorbent probes.

32. The kit of claim 28, wherein the probes are configured to absorb about 1-7 microliters of blood.

33. The kit of claim 28, wherein at least one of the probes contains dried blood.

34. The kit of claim 28, wherein at least one of the probes contains a dried anticoagulant.

35. The kit of claim 28, wherein at least one of the compartments contains a desiccant.

36. A method of forming an absorbent probe for absorbing liquids, comprising:
pyrolyzing a high carbon precursor to form a porous, open cell, carbonized probe;
fastening the probe to a holder configured to allow a user to manually manipulate the probe and holder during use.

37. The method of claim 36, wherein the carbonized probe has about 95% or more of the absorbent probe made of carbon, by weight.

38. The method of claim 36, wherein the carbonized probe has about 95% or more of the absorbent probe made of carbon, by weight.

39. The method of claim 37, further comprising absorbing an anticoagulant into the absorbent probe.

40. The method of claim 36, wherein the carbonized probe has a porosity of about 40%.

41. A fluid collection device, comprising:
a holder having a body configured to be gripped and manipulated by a person's hand and having a distal end with distal tip thereon, the holder having a longitudinal axis extending along a length of the holder;
an absorbent probe connected to the distal tip, the distal tip having a sidewall and end forming a continuous surface with the sidewall of the absorbent probe encircling a portion of the distal tip and the end of the absorbent probe forming a closed bottom of the recess, the absorbent probe end and sidewall having a substantially uniform thickness and an exterior surface with no concave portions thereon.

42. The fluid collection device of claim 41, wherein the absorbent probe is made of porous carbon.

43. The fluid collection device of claim 41, wherein the absorbent probe is made of sintered plastic.

44. The fluid collection device of claim 41, wherein the absorbent probe has a bullet shape with generally parallel sidewalls and a hemi-spherical end.

45. The fluid collection device of claim 41, wherein the absorbent probe has a conical shape.

46. The fluid collection device of claim 41, wherein the absorbent probe has a bullet shape with generally parallel sidewalls and a curved end having a conical surface forming at least a portion of that end.

47. The fluid collection device of claim 41, wherein the absorbent tip comprises absorbent means configured for absorbing a predetermined volume of fluid.

48. The fluid collection device of claim 41, wherein the sidewall of the absorbent tip has a thickness of about 0.015 to 0.063 inches.

49. The fluid collection device of claim 41, wherein the sidewall of the absorbent probe has a thickness of about 0.015 inches.

50. The fluid collection device of claim 43, wherein the absorbent probe comprises a non-porous inner portion connected to the distal tip and a porous outer portion.
